(19) United States
(12) Patent Application Publication
(10) Pub. No.: US 2019/0261888 A1
(43) Pub. Date: Aug. 29, 2019

(54) RE-WEARABLE WIRELESS DEVICE

(71) Applicant: Proteus Digital Health, Inc., Redwood City, CA (US)

(72) Inventors: Mark Zdeblick, Portola Valley, CA (US); Kien Lam, San Jose, CA (US); Jeremy Frank, San Francisco, CA (US); Robert Azevedo, Albany, CA (US); Elizabeth Klein, San Francisco, CA (US); David O'Reilly, Palo Alto, CA (US); Aditya Dua, San Jose, CA (US); Joan Hain, San Carlos, CA (US); Robert Leichner, Menlo Park, CA (US); Christopher Reggiardo, Castro Valley, CA (US); Lawrence Arne, Palo Alto, CA (US); Arna Ionescu Stoll, Seattle, WA (US); Kristin Pollock, Portland, OR (US); Olivier Colliou, Los Gatos, CA (US); James Fish, Redwood City, CA (US)

(21) Appl. No.: 16/405,324

(22) Filed: May 7, 2019

Related U.S. Application Data
(63) Continuation of application No. 15/147,593, filed on May 5, 2016.
(60) Provisional application No. 62/159,209, filed on May 8, 2015.

(51) Int. Cl. A61B 5/07; A61B 5/00; A61B 5/11

(52) U.S. Cl. CPC A61B 5/073; A61B 5/7455; A61B 2560/0219; A61B 2560/0242; A61B 5/1112

(57) ABSTRACT

A re-wearable wireless device configured to be attached to a user is disclosed. In some embodiments, the re-wearable wireless device includes a disposable component and a reusable component. The disposable component may include a first electrode, a second electrode, a cradle, and a battery. The cradle may include a first electrical connector, wherein the first electrical connector is electrically coupled to the first electrode, the second electrode, and the battery. The reusable component may include an electronics module, a housing configured to latch into the cradle, a second electrical connector, wherein the second electrical connector electrically couples the reusable component to the first electrode, the second electrode, and the battery. The re-wearable wireless device may be configured to detect a conductive electrical signal through the first electrode and the second electrode.

### CROSS REFERENCE TO RELATED APPLICATIONS

This application is a continuation application claiming priority under 35 U.S.C. § 120 to U.S. patent application Ser. No. 15/147,593, titled LOOSE WEARABLE RECEIVER SYSTEMS, filed May 5, 2016, which application claims the benefit under 35 U.S.C. § 119(e) of U.S. Provisional Application No. 62/159,209, titled LOOSE WEARABLE RECEIVER SYSTEMS, filed May 8, 2015, the disclosure of which applications are herein incorporated by reference in their entireties.

### INTRODUCTION

The present disclosure is related generally to loose wearable receiver systems. Loose wearable receiver systems are operable to detect, records, and/or transmit physiological information about a wearer of the system. In some aspects, a wearable system is operable to detect an electrical current signature produced by an ingestible device indicator system. In some aspects, the wearable system is operable to detect and monitor physiological parameters of the wearer, such as heart rate, heart rate variability, fluid level, hydration state, and/or temperature, among others. In some aspects, the wearable system is operable to detect and monitor activities of the wearer, such as physical activity, sleep and wakefulness, gait, falling, location, and/or stress, among others. The wearable system may be operable to analyze data gathered from the wearer, provide feedback to the wearer, store the wearer's data on a remote system, and/or transmit the wearer's data to caregivers. The wearable system is thus capable of functioning as a whole health system for the wearer. The wearable system may be configured to be interactive, attractive, fun, and/or desirable in order to encourage wear and use.

### SUMMARY

In some embodiments, a wearable system, is disclosed. The wearable system comprises a loose wearable component, configured to be removably worn attached to a user. The loose wearable component comprising a compartment configured to removably receive an electronics module and a battery-operated electronics module configured to be removably attached to the compartment. The loose wearable component is configured to detect an electrical current signature produced by an ingestible device when the ingestible device is located within a body of a user that wears the wearable component. The loose wearable component is configured to determine information from the electrical current signature. The information is related to a physical environment of the ingestible device.

In some embodiments, a device comprising a loose wearable component configured to be removably worn is disclosed. The loose wearable component comprises a compartment configured to removably receive an electronics module configured to detect an electrical current signature produced by an ingestible device.

In some embodiments, a device comprising an electronics module is disclosed. The electronics module is configured to be removably attached to a loose wearable component. The electronics module is configured to detect an electrical current signature produced by an ingestible device.

### FIGURES

The novel features of the embodiments described herein are set forth with particularity in the appended claims. The embodiments, however, both as to organization and methods of operation may be better understood by reference to the following description, taken in conjunction with the accompanying drawings as follows:

FIG. 1A illustrates one embodiment of a wearable system comprising a body-associated personal communicator configured to be worn by a person, herein referred to as the wearer;

FIG. 1B illustrates one embodiment of a device comprising a loose wearable component 102 configured to be removably worn;

FIG. 1C illustrates one embodiment of a charging station;

FIG. 2A illustrates a wearable component configured as an armband, and an electronics module configured to be removably attached to the armband;

FIG. 2B illustrates the wearable system with a wearable charging station;

FIG. 3 illustrates a cutaway view of the electronic module of one embodiment of the wearable system;

FIGS. 4A-4B illustrate one embodiment of the wearable system configured as a necklace and pendant;

FIGS. 5A-5B illustrate one embodiment of the wearable system configured as a pendant and counterweight;

FIGS. 6A-6B illustrate one embodiment of the wearable system configured as a shoulder strap;

FIGS. 7A-7B illustrate one embodiment of the wearable system configured as a tether;

FIG. 8 illustrates one embodiment of a wearable system configured as a clip;

FIG. 9 illustrates one embodiment of a wearable system configured as a neckband;

FIG. 10 illustrates one embodiment of a wearable system configured as a waistband;

FIG. 11 illustrates one embodiment of a wearable system configured as a necklace comprising an electronics module configured as a pendant;

FIG. 12 illustrates one embodiment of a wearable system configured as a waist or chest band;

FIG. 13 illustrates one embodiment of a wearable system configured as one or two armbands;

FIG. 14 illustrates one embodiment of an ingestible device event indicator (e.g. ingestible even marker or IEM) system with dissimilar metals positioned on opposite ends of a framework;

FIG. 15 illustrates one embodiment of an ingestible device indicator system with dissimilar metals positioned on the same end of a framework;

FIG. 16 illustrates one embodiment of the system of FIG. 14 in an activated state and in contact with conducting liquid;

FIG. 17 shows an exploded view of the surface of the first material;

FIG. 18 illustrates a block diagram representation of one embodiment of the control device of FIGS. 14 and 15;

FIG. 19 illustrates one embodiment of a system that includes a pH sensor module connected to a material, where the material is selected in accordance with a specific type of sensing function being performed.

FIG. 20 illustrates one embodiment of a personal communication system;

FIG. 21 illustrates a functional block diagram of one embodiment of a signal receiver employing a coherent demodulation protocol to read a packet of data present in a signal;

FIG. 22 illustrates a functional block diagram of a beacon module;

FIG. 23 illustrates a block functional diagram of one embodiment of an integrated circuit component of a signal receiver;

FIG. 24 illustrates a more detailed block diagram of one embodiment of a circuit configured to implement the block functional diagram of the signal receiver depicted in FIG. 23;

FIG. 25 illustrates a block diagram of hardware of a signal receiver according to one embodiment related to the high frequency signal chain; and

FIG. 26 illustrates an example of a system according to the disclosed embodiments.

FIG. 27 is a perspective view of the re-wearable wireless device with a removable liner removed from an adhesive layer, according to one embodiment.

FIG. 28 is a top view of the re-wearable wireless shown in FIG. 27, according to one embodiment.

FIG. 29 is an explode view of the reusable component of the re-wearable wireless device shown in FIG. 27, according to one embodiment.

FIG. 30 is an illustration of a perspective view of the reusable component 1402 and disposable component of the re-wearable wireless device shown in FIG. 27 prior to mating the two components, according to one embodiment.

FIG. 31 is a side view of the reusable component and disposable component of the re-wearable wireless device shown in FIG. 27 prior to mating the two components, according to one embodiment.

FIG. 32 is a side view of the reusable component and disposable component of the re-wearable wireless device shown in FIG. 27 after mating the two components, according to one embodiment.

FIG. 33 is a detail view of the electrical contact elements located within the re-usable component housing of the re-wearable wireless device shown in FIG. 27, according to one embodiment.

FIG. 34 is side view of the electrical contact elements within a re-usable component housing of the re-wearable wireless device shown in FIG. 27, according to one embodiment.

### DESCRIPTION

Before explaining the various aspects of the loose wearable receiver system in detail, it should be noted that the various aspects disclosed herein are not limited in their application or use to the details of construction and arrangement of parts illustrated in the accompanying drawings and description. Rather, any disclosed aspect of loose wearable receiver system may be positioned or incorporated in other aspects, variations, and modifications thereof, and may be practiced or carried out in various ways. Accordingly, aspects of loose wearable receiver system disclosed herein are illustrative in nature and are not meant to limit the scope or application thereof. Furthermore, unless otherwise indicated, the terms and expressions employed herein have been chosen for the purpose of describing the aspects for the convenience of the reader and are not to limit the scope thereof. In addition, it should be understood that any one or more of the disclosed aspects, expressions of aspects, and/or examples thereof, can be combined with any one or more of the other disclosed aspects, expressions of aspects, and/or examples thereof, without limitation. As used throughout this description, a loose device can be worn loosely by a user without the fixedly attaching the device to the user's body using adhesives or other fasteners, enabling the user to readily put on or remove the loose device.

In the following description, like reference characters designate like or corresponding parts throughout the several views. Also, in the following description, it is to be understood that terms such as front, back, inside, outside, top, bottom and the like are words of convenience and are not to be construed as limiting terms. Terminology used herein is not meant to be limiting insofar as devices described herein, or portions thereof, may be attached or utilized in other orientations. The various aspects will be described in more detail with reference to the drawings.

It will be appreciated that the term “medication” or “dose form” as used throughout this disclosure includes various forms of ingestible, inhalable, injectable, absorbable, topical or otherwise consumable medicaments and/or carriers therefor such as, for example, pills, tablets, capsules, gel caps, patches, placebos, over capsulation carriers or vehicles, herbal, over-the-counter (OTC) substances, supplements, prescription-only medication, ingestible event markers (IEM), and the like.

In one aspect, the present specification provides an ingestible device event indicator, also called an ingestible event marker or IEM. In one aspect, the ingestible device event comprises an activation component, a signal generation component, and additional components as desired. In one aspect, the activation component comprises dissimilar materials positioned on a framework. The activation component is activated upon ingestion of the ingestible device event indicator. Upon activation, the signal generation component produces a unique current signature that signifies that the ingestible device indicator has been ingested. The ingestible device event indicator can be used in association with any pharmaceutical product to determine when the patent takes the pharmaceutical product.

In one aspect, the present specification provides a body-associated personal wearable communication device (“body-associated personal communicator”). In one aspect, the body-associated personal communicator is in communication with a living subject. In one aspect, the body-associated personal communicator is in communication with a local node external to the body of the living subject. In one aspect, the local node is in communication with a remote node via a network and, accordingly, the living subject is able to communicate with the remote node. Information also may be communicated from the remote node and/or the local node to the living subject via the body-associated personal communicator. In various aspects, the two-way communication between the living subject and the body-associated personal communicator occurs discreetly, such that the communications are non-detectable by humans. Such discreet mode of communication minimizes the intrusiveness into the living subject's sense of privacy and enhances the likelihood that the living subject will accept the personal communicator and use it in a prescribed manner.

In one aspect, the present specification provides a body-associated personal communicator that senses personal physiologic parameters of the living subject—such as for instance the unique current from an ingestible device event indicator—and communicates such parameters to the local node and in some aspects to the remote node. Information associated with the personal physiologic parameters also may be communicated from the remote node and/or the local node to the living subject via the body-associated personal communicator. As described above, communications between the individual and the body-associated personal communicator occurs discreetly to enhance the likelihood of acceptance of the body-associated personal communicator by the living subject.

### Functionality

In various aspects, the body-associated personal communicator is operable to provide methods of functionality. In some aspects, the body-associated personal communication provides functionality to the wearer of the body-associated personal communicator, including but not limited to the ability to alert functions, authentication functions, detection functions, encouragement functions, feedback functions, informing functions, notification functions, protection functions, recognition functions, recording functions, sensing functions, sharing functions, and tracking. In some aspects, the body-associated personal communicator is operable to provide functional features, such as for instance automation functions, appearance functions control functions, display functions, docking functions, lighting functions, play functions, and wearability functions. In some aspects, the body-associated personal communicator is operable to provide data functions, such as for instance analytical functions and capture functions. In some aspects the body-associated personal communicator is operable to provide functionality that interacts with other things, such as for instances automation functions, communication functions, connectivity functions, delivery functions, detection functions, interference functions, notification functions, pay functions, recording functions, replacement functions, seeking functions, sensing functions, synchronization functions, and tracking functions.

In some aspects, the body-associated personal communicator is operable to provide functionality to the wearer of the body-associated communicator. In some aspects, the body-associated personal communicator may be operable to keep the wearer awake, allow the wearer to alert others that the wearer is suffering an emergency, and/or authenticate the identity of the wearer using a method such as bioauthentication. The body-associated personal communicator may further be operable to detect the alertness of the wearer, whether the wearer is currently possessing contraband articles or is smoking cigarettes or some other smokeable item, and/or is coughing. The body-associated personal communicator may further be operable to detect dual heartbeats in a pregnant wearer, fever, a fall by the wearer, the wearer's glucose level, an IEM ingested by the wearer, and/or whether the wearer is lying. The body-associated personal communicator may further be operable to detect whether the wearer is conducting a specific activity or any activity, and provide encouraging feedback to the wearer. The body-associated personal communicator may further be operable to provide biofeedback, that is, information about the wearer's physiological functions. The body-associated personal communicator may further be operable to inform the wearer of his or her proper breathing rate, such as for instance by blinking alight at the proper breathing rate. The body-associated personal communicator may further be operable to inform the wearer what wearer should eat based on the wearer's caloric intake and/or what food sources are nearby. The body-associated personal communicator may further be operable to notify the wearer with customized reminders, and/or that the wearer is outside of a specified area. The body-associated personal communicator may further be operable to provide the wearer with turn-by-turn directions. The body-associated personal communicator may further be operable to inform the wearer of the pharmacokinetics of any drugs the wearer is taking or has taken. The body-associated personal communicator may further be operable to inform the wearer as to his or her posture, and whether the wearer's posture should be corrected at a given moment. The body-associated personal communicator may further be operable to inform the wearer that the wearer is conducting an activity at an inappropriate time. The body-associated personal communicator may further be operable to monitor the wearer's sleep and notify the wearer with an alarm. The body-associated personal communicator may further be operable to protect the wearer with pepper spray or another protection device, such as a Taser. The body-associated personal communicator may further be operable to recognize speech. The body-associated personal communicator may further be operable to record electroenchephalography (EEG), electromyography (EMG), and/or other data about the wearer, as well as voice reminders and appointments. The body-associated personal communicator may further be operable to sense the wearer's blood flow, blood alcohol level, breathing rate, muscle recovery, over eating or over drinking, and/or stress level. The body-associated personal communicator may further be operable to sense external factors near the wearer, such as the air pressure around the wearer and/or the wearer's altitude. The body-associated personal communicator may further be operable to share information about the wearer, such as the wearer's biolevels. The body-associated personal communicator may further be operable to track the wearer's activity level, diet, food intake, GPS location (such as for geocaching or subway racing), loyalty to a program, microlocation, mood swings, addictions, sleep, and/or location using a system such as iBeacons (™)

In some aspects, the body-associated personal communicator is operable to provide functional features. In some aspects, the body-associated personal communicator may provide automation functions, such as automatically turning itself on and off at the appropriate times. In some aspects, the body-associated personal communicator may be further provide appearance functions, such as changing its appearance when the wearing is moving a great deal, an or changing color with the wearer's mood. In some aspects, the body-associated personal communicator may further provide control functions, such as providing a method to manually turn the communicator on and off. In some aspects, the body-associated personal communicator may further provide display functions, such as a dashboard-style display and/or displaying photographs. In some aspects, the body-associated personal communicator may further provide docking functions, such as docking to a charging device, docking to another device, and/or docking to a network. In some aspects, the body-associated personal communicator may further provide lighting functions, such as providing ambient light and/or flashlight-type focused light. In some aspects, the body-associated personal communicator may further provide play functions, such as playing music files, podcasts, and/or other streaming audio and/or video. In some aspects, the body-associated personal communicator may further provide wearability functions, such as being comfortably wearable for twenty-four hours, being integrated into an article that the wearer already wears, being wearable on any part of the body and moveable to another part of the body at any time, and being sufficiently versatile that it is not constrained to what is currently fashionable.

In some aspects, the body-associated personal communicator may provide data functions. In some aspects, the body-associated personal communicator may provide analytical functions, such as analyzing data detected, captured, and/or sensed from the wearer. n some aspects, the body-associated personal communicator may further provide capture functions, such as capturing physiological vital signs of the wearer, photographs, continuous video, and/or continuous audio.

In some aspects, the body-associated personal communicator may provide functionality that interacts with other things. In some aspects, the body-associated personal communicator may provide automation functions, such as automatically unlocking doors and/or identifying the wearer to the door being unlocked. In some aspects, the body-associated personal communicator may further provide communication functions, such as instant messaging, telephone, text messages, and/or instantaneous communication such as provided by a walkie-talkie. In some aspects, the body-associated personal communicator may be operable to act as a replacement to a cellular phone or a mobile phone. In some aspects, the body-associated personal communicator may provide communication to a social network. In some aspects, the body-associated personal communicator may provide connectivity functions, such as a W-Fi hotspot. In some aspects, the body-associated personal communicator may provide delivery functions, such as drug delivery to the wearer. In some aspects, the body-associated personal communicator may provide detection functions, such as metal detection, detection of nearby motion, detection of nearby smoke, carbon monoxide, or radioactivity. In some aspects, the body-associated personal communicator may provide interference functions, such as interfering or jamming nearby cellular or mobile phone signals. In some aspects, the body-associated personal communicator may provide notification functions, such as notifying a third party that the wearer is inside or outside a specific area. In some aspects, the body-associated personal communicator may provide pay functions, such as paying for an item when in proximity of a payment system, such as a cash register. In some aspects, the body-associated personal communicator may provide recording functions, such as recording audio or video. In some aspects, the body-associated personal communicator may provide replacement functions, such as replacing a key such as a car key, or a cellular or mobile phone. In some aspects, the body-associated personal communicator may provide seeking functions, such as seeking individuals with similar characteristics as the wearer, or seeking anyone who is near to the wearer. In some aspects, the body-associated personal communicator may provide sensing functions, such as sensing whether other wearable users are nearby, whether other wearable users are not nearby, and/or whether the wearer is alone. In some aspects, the body-associated personal communicator may provide synchronization functions, such as synchronization with another device such as a mobile device and/or computer. In some aspects, the body-associated personal communicator may provide tracking functions, such as tracking the current time.

### Methods of Attachment to the Body

In various aspects, methods of attaching the body-associated personal communicator to the wearer are disclosed. In some aspects, the body-associated personal communicator maybe attached to another article or to the wearer by various methods. In some aspects, the body-associated personal communicator maybe attached to certain parts of the wearer's body. In some aspects, the body-associated personal communicator maybe attached to an article carried by the wearer. In some aspects, the body-associated personal communicator maybe attached to an article used by the wearer. In some aspects, the body-associated personal communicator maybe attached to an article that is worn by the wearer.

In some aspects, the body-associated personal communicator maybe attached to another article or to the wearer by various methods. In some aspects, the body-associated personal communicator may be attached to another article, such as clothing, by an adhesive or glue, by hanging it to the article, by weaving it into the article, by inserting it into the article, by sewing it into the article, by tying it to the article, and/or by tucking it into the article. In some aspects, the body-associated personal communicator may be attached to the wearer by implanting it in the wearer, having the wearer ingest it, by subdermal magnets, and/or by weaving it into the wearer's hair. In some aspects, the body-associated personal communicator may be attached to the wearer by incorporating it into, or forming it in the shape of, implantable jewelry, an ingestible badge, a pacemaker, and/or subcutaneous implants.

In some aspects, the body-associated personal communicator maybe attached to certain parts of the wearer's body, such as around the ankle, in or on a beard, behind the ear, on or around the chest, in or on a mustache, on or over scars, similar to or as silica implants, as permanent or removable tattoos, inserted in the nostrils, or as a piercing.

In some aspects, the body-associated personal communicator maybe attached to or incorporated in an article carried by the wearer, such as car keys, mobile device attachments or accessories, mobile device holsters, mobile device cases, cellphone cases, credit cards, wallets, key chains, keys, mobile phones or other mobile devices, backpacks, or bag straps.

In some aspects, the body-associated personal communicator maybe attached to an article used by the wearer, such as a car steering wheel, somewhere in a car, in or on a pill bottle, in food in an ostomy bag, in an insulin pump, in or on a variety of things that may be touched by the wearer (such as bath scale, glass, phone, and/or anything else commonly around the wearer), in or on a chair, and/or in or on a pillow.

In some aspects, the body-associated personal communicator maybe attached to or incorporated into an article that is worn by the wearer. In some aspects, the body-associated personal communicator may be attached to or incorporated into clothing, such as a belt, a waistband, a hat, headbands, a jock strap, a neck tie, pants, shoe inserts, shoelaces, shoes, socks, outer clothing, suspenders, underwear, and/or a zipper tab. In some aspects, the body-associated personal communicator may be attached to or incorporated into an accessory, such as ear muffs, fashion pins, gloves, a lapel pin, a scarf, and/or a wrist band. In some aspects, the body-associated personal communicator may be attached to or incorporated into a piece or multiple pieces of jewelry, such as a belly button plug, a bracelet, an earring, a nose ring, a gold chain, a necklace, a pendant, a ring, a toe ring, and/or a wristwatch. In some aspects, the body-associated personal communicator may be attached to or incorporated into bag or pouch-type object carried by the wearer, such as fanny pack, a holster, a backpack, a purse, a purse strap, and/or a waist pack. In some aspects, the body-associated personal communicator may be attached to or incorporated into a medical device, such as eyeglasses, a back brace, other braces, a chest strap, compression arm bands, contact lenses, a hearing aid, a heart monitor, a knee braces, and/or a prosthetic. In some aspects, the body-associated personal communicator may be attached to or incorporated into a hair accessory, such as a bobby pin, a hair clip or pin, a hair tie or band, hairpins, and/or a wig or hairpiece. In some aspects, the body-associated personal communicator may be attached to or incorporated into a beauty product, such as artificial nails, and/or fingernail polish. In some aspects, the body-associated personal communicator may be attached to or incorporated into a dental appliance, such as braces or dentures. In some aspects, the body-associated personal communicator may be attached to or incorporated into an infant-related article, such as a diaper, a bib, and/or a baby carrier. In some aspects, the body-associated personal communicator may be attached to or incorporated into a piece of technology worn by the wearer, such as earpieces, headphones, and/or a Bluetooth (™) headset. In some aspects, the body-associated personal communicator may be attached to or incorporated into a work-related item, such as a professional badge or a company badge. In some aspects, the body-associated personal communicator may be attached to or incorporated into a safety or sports article, such as a helmet and/or other protective gear. In some aspects, the body-associated personal communicator may be attached to or incorporated into a legally required article, such as a movement tracker typically worn around the ankle. In some aspects, the body-associated personal communicator may be attached to or incorporated into something worn on the skin, such as pasties, temporary tattoos, and/or perfumes.

### Methods of Attaching Electrodes to the Wearer

In various aspects, the body-associated personal communicator comprises one or more electrodes that are attached to the wearer. In various embodiments, methods for attaching the electrodes to the body-associated personal communicator are disclosed. In some aspects, the electrodes may be attached by adhesives, liquids, mechanical fasteners, shaping the components a particular way, movement of the wearer, incorporation into an article worn by the wearer, use of a signal, or replacement of the electrodes.

In some aspects, electrodes may be fixed to the body-associated personal communicator by use of adhesives, such as chewing gum, conductive adhesive, conductive aerosol or hairspray or some kind of spray, conductive Play-Doh (™), denture cream, lamination, adhesive sprays, and/or stick on substances.

In some aspects, the electrodes may be fixed to the body-associated personal communicator by use of liquids, such as conductive liquid.

In some aspects, the electrodes may be fixed to the body-associated personal communicator by use of mechanical fasteners, such as heat treatment, bolts and nuts, laser welding, magnets, nails, screws, sewing on or in, snaps, solder, springs, sutures, ties, ultrasonic welding, Velcro (™), and/or conductive fabric.

In some aspects, the electrodes may be fixed to the body-associated personal communicator by shaping the components a particular way. In some aspects, the electrodes may comprise a flex circuit. In some aspects, the electrodes may be shaped as plugs and the body-associated personal communicator may comprise receptacles for such plugs. In some aspects, the electrodes may screw into or snap into the body-associated personal communicator.

In some aspects, the electrodes operate by the movement of the wearer. In some aspects, the electrodes operate as the wearer changes body positions and/or touches different parts of his or her body. In some aspects, the electrodes operate by touching a circuit, such as the electrodes and/or the body-associated personal communicator.

In some aspects, the electrodes are incorporated into an article worn by the wearer, such as a coat button, eye glasses, headphones, headsets, human and/or animal hair, a SIM card, and/or conductive fabric.

In some aspects, the electrodes are connected to the body-associated personal communicator by use of a signal, such as an inductive signal such as a wireless charging coil, optical signal, a wireless signal, and/or an ultrasound signal, and or an ultrasound signal sent through the body from part to another. In some aspects, the electrodes are activated by remote sensors.

In some aspects, the electrodes are replaced with capacitive sensing.

### Size Constraints

In various aspects, the body-associated personal communicator may be sized to meet certain size constraints, such as physical, technological, wearability, psychological, economic, and liability constraints.

In some aspects, the body-associated personal communicator may be sized to meet certain physical constraints, such as the ability to adhere to the wearer or another article, the ability to function at all angles, whether it has a button, the size and shape of a circuit board it may contain, the distance of the dipole between the electrodes, the amount and/or size of on-board electronics if present, the size and shape of an enclosure, wearer ergonomics, necessary hardware, the capability of hydrogels if used, the size, shape, and/or color of LEDs if present, whether it is a one piece device, the type and quality of any adhesive used, whether it is water resistant or waterproof, and its weight.

In some aspects, the body-associated personal communicator may be sized to meet certain technological constraints, such as accuracy of detection possible, the capability of batteries, IEM detection, jiggle, it' lifetime, that is, the amount of time it is to operate, manufacturing ability, material composition, on board memory capability, the limitations of physics, protection accuracy, signal strength, and/or the capability of today's technology.

In some aspects, the body-associated personal communicator may be sized to meet certain wearabilty constraints, such as comfort, handiblity, lack of itchiness, manageabilty by the wearer, the difference in the size and shape of wearers, wearability during sleep, and usability.

In some aspects, the body-associated personal communicator may be sized to meet certain psychological constraints, such as being discreet and/or not embarrassing to wear.

In some aspects, the body-associated personal communicator may be sized to meet certain economic constraints, such as what as competition, cost, and/or the number features.

In some aspects, the body-associated personal communicator may be sized to meet certain liability constraints, such as electrical safety.

### Size Reduction

In various aspects, the body-associated personal communicator may be made small in size according to various methods, such as how it is built, how it operates, the impression it makes on the wearer, and/or how it operates. In various aspects, the need for it to be small may be obviated by, changing its appearance or increasing the cost.

In some aspects, the body-associated personal communicator may be made small by how it is built. In some aspects, the body-associated personal communicator may be built with small distributed parts; built using chip-on-flex or a flex circuit to remove hard pieces; built with different pieces for different functionalities; built to be disposable daily, built flat and integrated into footwear; made flexible; made low-profile and thus hide-able under or in clothing; built with a formable battery as the enclosure; built with a fully-custom ASIC; built with invisible adhesive; built in the shape of a spear; built lightweight; made with interchangeable parts; built such that the battery is the enclosure; make with interchangeable parts to minimize functions; built with a thin metal enclosure; built with minimal connector size by integrating connectors into the structure of components; made modular; built without an enclosure or a mobile device; built without plastics; built with all components separated; built as small, distributed parts; built with smaller radios; built with snap-in parts; built in he shape of a sphere; built with thin-film batteries; and/or built with a water-sealed printed circuit board.

In some aspects, the body-associated personal communicator may be made small by how it works, such as by more sensitive IEM detection, employing extra low-energy Bluetooth (™), integrating connections into the structure of the components, using optical and/or conductive communication methods instead of radio, reducing the length of the dipole, reducing the size and lifetime of the battery, and/or removing any flash memory.

In some aspects, the body-associated personal communicator may be made small by how it charges, such as employing a bedside automatic charger, charging it by having the wearer touching every day things such as anything the wearer sits on (i.e. a chair) or in (i.e. a car), charging it with body movements, employing conductive charging, docking when the wearer sits on something such as chair, drawing power inductively from another device such as a mobile device, employing portable batteries, employing rechargeable batteries, using a wireless charging mat and optionally having the device enter a sleep mode while resting on the mat, employing solar power, using the device's temperature as a delta for power, and/or using a wired power source.

In some aspects, the body-associated personal communicator may be made small by the impression it makes on the wearer, such as its attractiveness, its inability to tangle, how ubiquitous it is, heightening the desire to wear it, and/or by not requiring that it be attached to the wearer.

In some aspects, the body-associated personal communicator may be made small by how it operates, such as eliminating permanently attached electrodes, reducing the data capture duty cycle, reduce the functionality, reduce pixels in the display, allowing it to be turned on by demand and instead of being always on, and/or by use a projection display

In some aspects, the need for the body-associated personal communicator to be small may be obviated by changing its appearance, such as using active camouflage, disguising it as something else, incorporating it into something that is already large, hiding it under the skin, disguising it as skin, and/or incorporating its components into every day items such as a mobile device, earring, and/or watch.

### Measurable Metrics

In various aspects, the body-associated personal communicator may be operable to measure a number of metrics associated with the wearer, such as physical metrics, mental metrics, environmental metrics, and lifestyle metrics.

In some aspects, the body-associated personal communicator may be operable to measure physical metrics of the wearer. In some aspects, the body-associated personal communicator may be operable to detect that the wearer has ingested an IEM. In some aspects, the body-associated personal communicator may be further operable to measure the wearer's balance, blood alcohol level, blood capacity, blood pressure, blood viscosity, brain waives, breathing, breathing pattern, fluid levels, caloric intake, and/or whether the wearer is suffering muscle cramps. In some aspects, the body-associated personal communicator may be further operable to measure how much energy the wearer has expended, as well as falls by the wearer and the wearer's current fertility. In some aspects, the body-associated personal communicator may be further operable to measure the wearer's gait, glucose level, heart rate, hydration level, hypertension, lung fluid level such as for congestive heart failure, menstrual cycle, and state of pregnancy. In some aspects, the body-associated personal communicator may be further operable to measure the side effects of drugs on the wearer, the wearer's sleep quality, sperm count, stress level, and sweat. In some aspects, the body-associated personal communicator may be further operable to conduct sweat analysis and/or measure the electrolytes in the wearer's sweat. In some aspects, the body-associated personal communicator may be further operable to measure the wearer's physical symptoms, temperature, fluid viscosity, water consumption, weight, and/or weight fluctuations.

In some aspects, the body-associated personal communicator may be operable to measure mental characteristics of the wearer, such as the wearer's chi flow, IQ, mindfulness, and/or mood.

In some aspects, the body-associated personal communicator may be operable to measure the lifestyle aspects of the wearer. In some aspects, the body-associated personal communicator may be operable to measure the wearer's activity level, caloric expectation, number of cigarettes smoked, diet, driving habits, food cravings, gaming status, life extension, life shortening, money saved, number of drinks drunk, and or strain.

In some aspects, the body-associated personal communicator may be operable to measure aspects of the wearer's environment, such as exposure to chemicals, the wearer's location, pollutants and/or distribution of pollutants in the vicinity, nearby smoke, and/or nearby toxicity.

### Heart Rate Detection

In various aspects, the body-associated personal communicator may be operable to detect the wearer's heart rate. In some aspects, the body-associated personal communicator may be operable to detect the wearer's heart rate using at least two electrodes.

In some aspects, the body-associated personal communicator may be operable to detect the wearer's heart rate by where it is worn, and/or by the wearer's actions. In some aspects, the body-associated personal communicator may be operable to detect the wearer's heart rate by methods not employing electrodes, and/or by employing a separate device.

In some aspects, the body-associated personal communicator may be operable to detect the wearer's heart rate b where it is worn, such as on the wrist or on the back of the neck, for example as a neck strap with electrodes therein.

In some aspects, the body-associated personal communicator may be operable to detect the wearer's heart rate by the wearer's actions, such as when the wearer touches the device.

In some aspects, the body-associated personal communicator may be operable to detect the wearer's heart rate by methods not employing electrodes, such as measuring the visual changes in skin hue, constriction and/or dilution, pressure in the mouth during breathing, pressure waves across the chest, and/or voice modulation. In some aspects the body-associated personal communicator may be operable to detect the wearer's heart rate by measuring the heart rate acoustically, auditorily, electrically, optically, by employing remote sensing such as sensing an electric field, employing ultra-wide band radar, and/or by measuring vasoconstriction or dilation.

### Small Motions by the Wearer

In various aspects, the body-associated personal communicator is operable to account for small motions that can occur when the device is loose attached to the wearer and the wearer moves, sometimes referred to as jiggling motions or the jiggle. In some aspects, the body-associated personal communicator is operable to account for small motions by compensating for the motions, by how the device is worn, by how the device is constructed, by employing personalization, by employing an additional device, by asking for feedback from the wearer, or by ignoring the small motions.

In some aspects, the body-associated personal communicator is operable to account for small motions compensating for the motions, such as being able to absorb the motions, by active cancellation based on body proximity, by averaging the motions over time, by correlate the motions with other measurements, by filtering the motions, and/or by modeling the motions in order to understand how the whole body is moving.

In some aspects, the body-associated personal communicator is operable to account for small motions by how the device is worn, such as having it be worn centered and connected to body angle, and/or by having it be worn on a stable anatomical position (i.e. the chest or back) and using that position as an offset to a reclining angle, by having it be worn in a location that is not prone to small motions (i.e. behind the ear), by having it be worn in a shoe, and/or by having it be worn under tight clothing.

In some aspects, the body-associated personal communicator is operable to account for small motions by how the device is constructed, such as constructing it to be sufficiently heave to not make small motions, by placing an accelerometer in the electrodes and place the electrodes stably on the wearer, by separating the device and the accelerometer and placing the accelerometer on a stable location on the wearer, by employing different accelerometers based on the wearer's level of activity, such as by employing different attachments, and/or by using only passive motion sensing.

In some aspects, the body-associated personal communicator is operable to account for small motions by employing personalization, such as compensating for body size (a smaller person has a smaller stride than a long-legged person), by machine learning, by training on morphology, by employing unique algorithms for each wearer to known when the device is jiggling and when it is not, and/or by training the device, such as by learning the wearer's walk.

In some aspects, the body-associated personal communicator is operable to account for small motions by employing an additional device. In some aspects, the body-associated personal communicator is operable to account for small motions by employing a location sensing device (i.e. GPS) to indicate that the device might be moving more because the wearer is moving faster. In some aspects, the body-associated personal communicator is operable to account for small motions by placing an accelerometer in a head-worn apparatus such as headphones, and/or by placing an accelerometer in the electrodes. In some aspects, the body-associated personal communicator is operable to account for small motions by combining the movements it measures with data from a mobile device. In some aspects, the body-associated personal communicator is operable to account for small motions by employing a gyroscope. In some aspects, the body-associated personal communicator is operable to account for small motions by analyzing other measured data. In some aspects, the body-associated personal communicator is operable to account for small motions by employing multiple sensors placed throughout the body, such as by examining if the sensors are moving together or independently. In some aspects, the body-associated personal communicator is operable to account for small motions by employing a gravity sensor to orient up and down.

In some aspects, the body-associated personal communicator is operable to account for small motions by asking for feedback from the wearer, such that the wearer can inform the device that he or she is causing jiggling.

In some aspects, the body-associated personal communicator is operable to account for small motions by ignoring the small motions, such as recognizing consistent motions that can be ignored, by distinguishing a jiggling motion from any other motion and ignoring the jiggling motion, and/or by abstracting the jiggling motion and employing a unique measurement of movement.

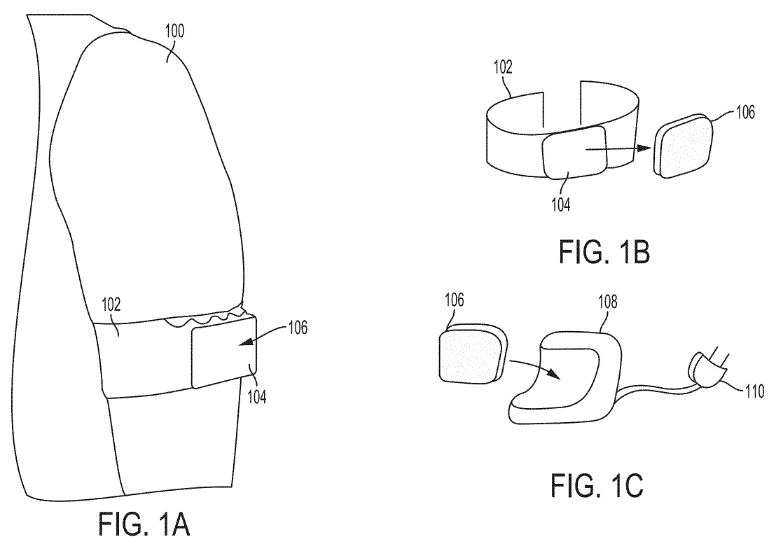

FIG. 1A illustrates one embodiment of a loose wearable system comprising a body-associated personal communicator configured to be loose worn by a person, herein referred to as the wearer 100. In some aspects, the wearable system comprises a loose wearable component 102 configured to be removably worn by the wearer 100 and a battery-operated electronics module 106. In one aspect, the loose wearable component 102 comprises a compartment 104. The electronics module 106 may be configured to be removably attached to the compartment 104. By removably attached is meant that the electronic module 106 may be placed in and/or attached to the compartment 104, using buckles magnets, snaps, springs, clips, clasps, buttons, screws, or any other appropriate fastener, and/or any combination of fastening methods and subsequently removed. Alternatively or additionally, the compartment 104 may be configured such that the electronics module 106 can be placed in and/or attached to the compartment 104 by press fit, tension fit, shoe-in, snap fit, twist, or any other appropriate locking method and/or any combination of locking methods and subsequently removed. Alternatively or additionally, the compartment 104 may comprise a pocket in which the electronics module 106 can rest. In one aspect, the wearable system is configured to detect an electrical current signature produced by an ingestible device, such as the ingestible device indicator system described in further detail below.

FIG. 1B illustrates one embodiment of a device comprising a loose wearable component 102 configured to be removably worn. In one aspect, the loose wearable component 102 comprises a compartment 104 that may be configured to removably receive an electronics module 106. Also illustrated is one embodiment of an electronics module 106 configured to be removably attached to a loose wearable component 102.

FIG. 1C illustrates one embodiment of a charging station 108. The charging station 108 is configured to recharge the battery of the electronics module 106. The charging station 108 may comprise a plug 110 for plugging into a wall socket. Alternatively or additionally, the charging station 108 may comprise a wireless charging module. In some aspects, the charging station 108 may comprise a box-shaped holder for the electronics module 106. In some aspects, the electronics module 106 automatically turns off when placed on or into the charging station 108, and turns on automatically when removed. In some aspects, the electronics module 106 may disable any on board radios when place on or into the charging station 108. In some aspects, the charging station 108 is configured to detect the presence of the electronics module 106 by employing a Hall effect sensor and/or a reed sensor and a magnet, wherein the magnet may be placed in either the electronics module 106 or the charging station 108, or vice versa. In some aspects, the charging station 108 incorporates a place to rest the loose wearable component 102, with or without the electronics module 106. In some aspects, the charging station incorporates a cleaning station, such as for instance a steam chamber, a pressure wash, an ultraviolet light, and/or an ultrasonic wash to clean the wearable component. In some aspects, the wearable system may comprise an enclosure for washing the loose wearable component 102 in a washing machine. In some aspects, the electronics module 106 can be washed along with the loose wearable component 102.

In some embodiments, the loose wearable component 102 comprises a band configured to be worn around a body limb, such as for example the lower arm, upper arm, calf, and/or thigh. The band may comprise a stretchable material, capable of expanding to accommodate the width of the limb and stay in place during all activities by the wearer 100. In some aspects, the band is non-constrictive and adjustable for all sizes and varieties of limbs. Alternatively or additionally, in some aspects the band may be provided in multiple sizes, with individual sizes being capable of adjustment for a range of sizes and varieties of limbs. In some aspects, the band is construction to be comfortable during high activity (such as strenuous exercise), moderate activity, and no activity (such as resting or sleeping). In some aspects, the band can be worn in all conditions, including full immersion, is able to wick away moisture from the wearer's body, and is breathable, meaning that is allows the skin of the wearer to breathe. In some aspects, the band is able to dry quickly if moistened, and may be cleaned with water and/or a washing detergent. In some aspects, the loose wearable component 102 may comprise an anti-bacterial material, and/or a stain-resistant coating.

In some embodiments, the loose wearable component 102 comprises one or more electrodes, wherein the electrodes make contact with the skin of the wearer 100 and are operable to detect information about the wearer. In some aspects, the loose wearable component 102 comprises two electrodes that form a dipole therebetween, operable to sense information about the wearer 100, such as for instance electrocardiography (ECG), and/or hear rate variability. In some aspects, the loose wearable component 102 comprises four electrodes, and is operable to sense for example the hydration status and/or fluid levels of the wearer 100. In such aspects, the electrodes can alternatively or additionally be employed in two pairs in order to more accurately sense the wearer's 100 heart rate. In some aspects, the electrodes are constructed of a material that does not require extra hydration, such as a hydrogel, in order to make sufficient contact with the skin of the wearer. In some aspects, the electrodes are constructed of an occluding material such that the wearer's 100 sweat will collect under the electrode to help maintain low impedance between the electrode and the skin. In some aspects, the electrodes are constructed of a material that is capable of drying without losing the moisture contact with the wearer's 100 skin. In some aspects, the electrodes are constructed of a material that is self-lubricating, such as for instance a material that absorbs skin oil to create a better contact with the skin, and/or a material that degrades to produce a bio-compatible material that maintains good contact as the material degrades. In such aspects, the electrode material may be sensitive to heat and/or pressure.

In some embodiments, the loose wearable component 102 comprises an electrically conductive, stretchable fabric. In such embodiments, the electrodes may comprise silver-silver chloride ink that has been printed or silk-screening or placed by MEMS deposition onto the electrically conductive fabric. One some embodiments, the loose wearable component 102 comprises vinyl impregnated with carbon and coated on the external side with silver ink and printed on the internal side with silver-silver chloride ink, such that the loose wearable component 102 is relatively waterproof. In some embodiments, the silver-silver chloride ink is printed in a specific pattern that achieves the best moisture contact with the skin.

In some embodiments, the electrodes are placed on or attached to the loose wearable component 102 to achieve the greatest possible distance between the electrodes, and thus the longest possible dipole formed by the electrodes. In one aspect, the electrodes are placed on opposite sides of the limb. In one aspect, the electrodes are placed on the front and the back of the limb. In one aspect, the electrodes are placed to be on opposite sides of the limb regardless of the size of the limb. In one aspect, the electrodes are placed independently of the location of the electronics module 106.

In some embodiments, the loose wearable component 102 comprises an electrically conductive stretchable fabric as previously discussed. In such embodiments, an electrical connection can be formed between any electrodes on or attached to the loose wearable component 102 and an electronics module 106. In one aspect, the compartment 104 comprises electrical contacts, such that when the electronics module 106 is placed in or attached to the compartment 106, the electronics module 106 is able to form an electrical connection with the compartment 104 and/or the loose wearable component 102. In some aspects, the compartment 104 facilitates the electrical connection by compression or clasping the electronics module 106 against the electrical contacts. In some aspects, the loose wearable component 102 comprises conductive paths and the electronics module 106 is configured to make an electrical connection with those paths. In some aspects, a ground plane is integrated into the loose wearable component 102 in a semi-permanent fashion so that the wearable component can be washed. In such embodiments, the electronics module 106 may establish two electrical connections, one to the ground plane and one to the electrodes.

In some embodiments, the electronics module 106 comprises one or more sensors. In some aspects, the one or more sensors may comprise one or more of a thermistor, an accelerometer, an ambient light sensor, a pressure sensor, a passive infrared sensor, and/or a gyroscope.

In some embodiments, the electronics module 106 comprises a thermistor, capable of measuring the body temperature of the wearer 100.

In some embodiments, the electronics module 106 comprises an accelerometer, operable to measure the acceleration of the wearer 100. The acceleration of the wearer can indicate how quickly the wearer 100 is moving and/or whether the wearer 100 has fallen. In some aspects, the wearable system is able to distinguish between a fall by the wearer 100 and some other possibly abrupt motion, such as driving over a speed bump.

In some embodiments, the electronics module 106 comprises an ambient light sensor, capable of measuring the amount of light in the vicinity of the wearer 100. In some embodiments, the wearable system is worn underneath clothing, and the ambient light sensor is operable to detect whether clothing has been removed and potentially whether the wearable system has been removed from the body.

In some embodiments, the electronics module 106 comprises a pressure sensor, operable to detect the atmospheric pressure in the vicinity of the wearer 100, and/or the pressure exerted by the wearer 100. In some aspects, pressure exerted by the wearer 100 may indicate an increased level of activity or a fall. In some aspects, the electronics module 106 comprises a blood pressure sensor. In such aspects, the loose wearable component 102 may be operable to inflate to create sufficient pressure on the wearer 100 in order to sense the wearer's 100 blood pressure. In such aspects, the loose wearable component 102 may comprise an inflatable component, or may be constructed of a piezoelectric material capable of being tightened to create sufficient pressure on the wearer 100 in order to sense blood pressure. In some aspects, the loose wearable component 102 is made from nitinol, and thus is operable to flex in order to provide sufficient pressure for a blood pressure reading. In such aspects, the loose wearable component 102 may further be coated with parylene as a moisture and dielectric barrier.

In some aspects, the wearable system comprises a module for measuring blood pressure that attaches to the loose wearable component 102. The module for measuring blood pressure may comprise, for example, a balloon that the wearer squeezes to inflate the loose wearable component 102 to create sufficient pressure to read blood pressure. In such aspects, the wearer 100 may attach the module for measuring blood pressure when he or she wishes to sense blood pressure. In such aspects, the electronics module 106 may be operable to read blood pressure in an automated fashion, and indicate to the wearer 100 when the reading has been taken. In some aspects, the module for measuring blood pressure may be incorporated in the charging station 108 such that when the wearer 100 attaches the wearable system to the charging station 108, the charging station 108 may initiate a blood pressure measurement.

In some embodiments, the electronics module 106 comprises a passive infrared (IR) sensor. In some aspects, the passive IR sensor is operable as a sociability sensor. In such aspects, the passive IR is operable to detect a warm body, and differentiate many pixels as being another person from a small number of pixels as indicating the presence of, for instance, a small animal.

In some embodiments, the electronics module 106 comprises a hydration sensor capable of sensing the fluid level of the wearer 100. In some aspects, the wearable system is operable to use a pitch-catch method to detect the fluid level of the wearer 100. The pitch-catch method comprises one set of electrodes for initiating a signal, placed in a first location, and a second set of electrodes for detecting the signal, placed at a second location. By employing at least two electrodes per set, the impedance of the skin can be bypassed, and only the impedance of the body can be measured. Additionally, the electrodes need not be damp for a proper measurement. In some aspects, different frequencies can be used to sense different tissue profiles; for instance, a higher frequency is capable of penetrating deeper under the tissue than a lower frequency. It is understood that body impedance correlates with body mas index (BMI) and thus that measuring body impedance can be used to indicate BMI.

In some embodiments, the electronics module 106 comprises a breathing sensor. In some embodiments, the loose wearable component 102 is comprises of a material that changes conductivity as it is stretched, such as for instance a piezoelectric film, which changes voltage as it is stretched; the change in conductivity may be uses to indicate the wearer's 100 breathing rate. The stretching and contraction of the loose wearable component 102 may also function as a strain gauge, indicating strain on the loose wearable component 102.

In some embodiments, the electronics module 106 comprises a microphone, operable to detect audible sounds in the vicinity of the wearer 100. In some aspects, the microphone can detect sleep apnea or other kinds of breathing issues. In some aspects, the microphone can listen for the wearer's 100 heart rate, and thus measure the wearer's acoustic heart rate. In some aspects, the microphone can function as a stethoscope, and provide a caregiver an audible indication of the wearer's heart rate. In some aspects, the microphone can be operable to detect stress.

In some embodiments, the electronics module 106 comprises a capacitive proximity sensor. In some aspects, the capacitive proximity sensor is operable to detect if the wearer 100 is currently wearing the wearable system. In some aspects, the capacitive proximity sensor can indicate to any onboard antennas that the wearer 100 is not wearing the wearable system, and thus that the antennas can be powered down.

In some embodiments, the electronics module 106 comprises one or more communications modules. In some aspects the one or more communications modules may comprise one or more of a Bluetooth (™) module, a cellular modem, a wireless antenna module, a Global Positioning System (GPS) module, or a Global Navigation Satellite System (GNSS) module.

In some embodiments, the electronics module 106 comprises a Bluetooth (™) module, operable to communicate to other devices by way of the Bluetooth (™) wireless protocol. Such other device may comprise, for example, a mobile device such as a smartphone or personal digital assistant, a desktop computer, a laptop, a charging station 108, or any other device that implements the Bluetooth (™) protocol.

In some embodiments, the electronics module 106 comprises a cellular modem. In some aspects, the cellular modem enables the electronics module 106 to communicate directly with the cellular network, including direct communication with the Internet. In some embodiments, the wearer's data is stored on a remote system, and the cellular modem allows the electronics module 106 to send and receive the wearer's data to and from the remote system. In some aspects, the cellular-enabled electronics module may be configured as a relay for Telehealth appliances. In some aspects, the electronics module 106 comprises an LTE modem. In such aspects, the electronics module 106 may function as an access point to the LTE network for other devices (commonly called a hotspot).

In some embodiments, the cellular modem and/or GPS module and/or GNSS module can be used to enable a geo-fence. A geo-fence is a designated area in which the wearer 100 is expected to be located. Geo-fences can be pre-designated areas, such as the wearer's 100 home, a family member's home, and/or a doctor's office. A caregiver may need to be informed when a wearer 100 has left the geo-fence, such as for instance if the wearer has Alzheimer's and tends to wander. Wearers who are self-sufficient may also want to inform a caregiver when he or she has left a geo-fenced area. Alternatively or additionally, the wearer 100 may be able to leave a geo-fence if accompanied by a caregiver. A cellular modem and/or GPS module and/or GNSS module may be configured to detect when the wearer 100 has gone outside the geo-fence, and the electronic module 106 can be configured to inform a caregiver and/or emergency personnel of this event. A cellular modem and/or GPS module and/or GNSS module may alternatively or additionally be configured to track the wearer 100 and detect or predict an emergency situation. In some aspects, the cellular modem and/or GPS module and/or GNSS module can be configured to give the last known location of the wearer 100, or periodically give the present location of the wearer 100, to assist in finding the wearer 100. In some aspects, leaving the geo-fence can trigger the electronics module 106 to make a noise or cry for help or flash lights in order to assist others in finding the wearer 100. In some aspects, the electronics module 106 can enable beacon functionality when the wearer exits the geo-fence, so that another device, such as a mobile phone, enabled to find the beacon can be employed to locate the wearer 100. In such aspects, a low-energy communication module, such as Bluetooth (™) can be employed to locate the wearer 100. Additionally or alternatively, a geo-fence can be employed to indicate areas in which the wearer 100 should not go. Alternatively or additionally, the electronics module 106 can be configured to indicate that the wearer 100 is with a certain person and is not wandering, even if the wearer 100 has gone beyond the geo-fence.

In some aspects, the GPS module and/or GNSS module can be configured to give the wearer 100 directions, either to return the wearer to a geo-fenced area, or to direct the wearer 100 to a location the wearer wishes to go. In some aspects, the electronics module 106 may be configured to accept voice input, such as “take me home.” In some aspects, the electronics module can give directions by haptic feedback, by audible directions, and/or by employing LEDs to indicate to the wearer 100 that the wearer 100 is heading in the correct direction (such as indicating “warm” or “cold”).

In some embodiments, the one or more communications modules can be configured to provide beacon functionality. A beacon area is an area configured to be a certain distance from a beacon. A beacon can, for instance, be placed in a charging station 108 for the electronics module 106, and indicate that the wearer 100 is within some distance of the charging station 108. In such aspects, the electronics module 106 may further be configured to avoid using functionality that requires more power or that do not work well within buildings. In some aspects, the electronics module 106 may be configured to use a low-energy communications module, such as Bluetooth (™) and/or Wi-Fi, to track that the wearer 100 is within the beacon zone. In some aspects, the beacon zone can be used to tether the wearer 100 to a temporary location such as a doctor's office.

In some embodiments, the electronics module 106 comprises at least one wearer interface. In some aspects, the wearer interfaces comprises one or more of an LED, a vibration motor, a touch sensor, or a tap sensor.

In some aspects, the electronics module 106 comprises one or more LEDs. In such aspects, the LEDs can be employed to glow in different colors for different situations. For example, the wearable system may be operable to analyze the wearer's 100 biometric data to determine a color for the day or the week or the moment, and/or giving feedback to the wearer 100 on how he or she is doing at any given moment. Alternatively or additionally, the LEDs can be used to personalize the wearable system according to the wearer's 100 tastes or desires. In some aspects, the LEDs can be employed to give the wearer 100 reassurance as to his or her current health state, to indicate adherence to medical routine, and/or whether the wearer 100 is within a geo-fence.

In some aspects, the electronics module 106 comprises a touch or tap sensor. In some aspects, the wearer 100 can tap the pod to ask the pod if it is operable. In such aspects, the electronics module 106 may respond with a light or vibration or voice to indicate that it is operable. In some aspects, the electronics module 106 may respond with more information, such as indicating that it is operable to not communicating with anything. In some aspects, the wearer 100 may be able to tap the pod as for game play. In such aspects, the user's response time may indicate the quality of the wearer's 100 reflexes. In such aspects, the electronics module 106 may vibrate in response to the wearer's 100 actions, and the wearer's 100 sensitivity to the vibration may be employed to indicate loss of nervous sensitivity. Game play functionality may be employed as a means of encouragement, such as encouraging the wearer 100 to take is or her medications, by rewarding adherence and/or progress. Game play may also provide reassurance by encouraging repetitive behavior, such as for autistic children. Game play may comprise simple memory games, using LEDs and vibrations. Memory games may assist those with neurodegenerative disorders, such as short term memory loss. Game play may also be employed to track a wearer' 100 mental decline or state of confusion. For example, if the wearer 100 achieves a score within a certain range, the wearer 100 may be feeling confused or having an adverse drug reaction.

In some aspects, the electronics module 106 comprises a speaker, operable to provide audible feedback to the wearer 100. In some aspects, the wearable system may employ an ambient light sensor to determine whether the wearable system is currently covered by clothing, and thus that the speaker would be muffled.

In some embodiments, one or more sensors can be used together to provide additional functionality. For example, a gyroscope and an accelerometer may be employed to take intense readings for a certain length of time every day, at the same time of day, to determine if the wearer's 100 walk or other physical behavior has changed over time. In such aspects, the wearable system may be employed to detect and/or monitor diseases involving motor functions, such as Parkinson's, or the onset thereof. In some aspects, impedance measurement of the skin can indicate skin turgor. Skin turgor is an indication of the level of hydration of the skin. By measuring skin turgor, the wearable system can measure the wearer's 100 hydration level. Data, such as impedance measurements, can be transmitted to the charging station 108 and further communicated to a caregiver. Impedance measurements can also be used to indicate that the wearable system is presently on the wearer's 100 body. In some aspects, the wearable system may be configured to record the wearer's 100 data over time, and correlate the data to the wearer's 100 overall health. For example, the wearer's 100 heart rate variability over time may indicate the wearer's stress levels. The data sensed by the wearable system may also be used to improve the usage of the wearable system. For example, by detecting the wearer's 100 blood flow, the wearable system may be able to determine that the loose wearable component 102 is being worn too tight, and inform the wearer 100 of this fact. In some aspects, sensors that indicate the wearer's 100 level of activity can be used to determine whether the wearer 100 is asleep or awake, and/or whether it is time for the wearer to take medication; if this is the case, the electronics module 106 may be operable to vibrate to inform the wearer 100 that it is time to take medication.

In some embodiments, one or more sensors can be used together to provide additional functionality, such as conserving battery usage. For example, in some aspects the timing and/or intensity of the vibration motor can timed and/or adjusted according to the wearer's 100 activity level (as measured, for example, by the accelerometer). In such aspects, the vibration motor may, for example, only activate when the wearer 100 is stationary or is exhibiting a minimal amount of activity—as lack of any activity may indicate that the wearer 100 is sleeping—since a very active wearer 100 may not be able to sense vibration. In some aspects, the vibration motor can be employed to wake a sleeping wearer 100 in a gradual way. In some aspects, the ambient light sensor can be employed to set the brightness of any LEDs, such that the LEDs are less bright when the local light is dimmer. In some aspects, the wearable system may be operable to determine that the wearer 100 is at home or sleeping; in such aspects, the wearable system may communicate with the charging station 108 such that the charging station 108 may take over battery-intensive operations, such as communicating by cellular modem or W-Fi to a remote system. In some aspects, the wearable system may alternatively or additionally communicate with a mobile device such that the mobile device may take over battery-intensive operations. In some aspects, the wearable system comprises a spare loose wearable component 102, such that the wearer 100 has an extra loose wearable component 102. In such aspects, the loose wearable component 102 that is not presently worn can be, for example, recharging a battery and/or be used for battery-intensive operations.

In some aspects, an accelerometer can be used to indicate how the wearer 100 is moving, such as whether the wearer 100 is in an airplane that is taking off. In such embodiments, a three-axis accelerometer may be employed to detect the state of the aircraft from takeoff to landing and automatically enter the electronics module 106 in an appropriate mode for each phase of flight (takeoff, climb, cruising, decent, landing) as defined by the regional regulations (laws) and the aircraft operator; such in-flight mode is sometimes referred to as airplane mode. Additional information obtained from a three-axis gyroscope (angular change) and from a three-axis magnetometer (compass) can refine the detection. Alternatively or additionally, in locales and with operators that allow its use, a GNSS receiver can be used as an alternate means to detect the aircraft states or to augment the information from the accelerometer. In some aspects the wearable system is configurable for which electronics and radios may be employed in given locale and or with each aircraft operator. In some aspects, the wearable system may be configured by a remote system through the cellular modem.

In some aspects, the wearable system is configured for takeoff of an aircraft. The takeoff period may be comprised, for example, of altitudes of fewer than 3,000 m (10,000 feet). The accelerometer may measure initial acceleration from low to high speed, such as for example from a speed of less than 40 km/h (25 MPH) to a speed of greater than 160 km/h (100 MPH). During takeoff, the wearable system may enter an initial mode, indicating that the wearer 100 may be in an airplane that is about to take off. In those mode, the wearable system may disable some functions as needed, such as the cellular modem, Bluetooth (™), GNSS, IS detection, gyroscope, and/or magnetometer. As the airplane's altitude increases, for example to greater than 100 m (328′), the wearable system may confirm that the wearer is in an airplane that is taking off. Sensing a further increase in altitude allows the wearable system to distinguish between an airplane taking off and, for example, acceleration as from a fast car, an aborted takeoff, or an amusement park. In the second phase of takeoff, the wearable system may enter airplane mode for takeoff, and disable necessary functionality.

During flight (e.g. at altitudes of greater than 3,000 m (10,000 feet)), the wearable system may enable functionality as is allowed by local regulations. For example, the wearable system may enable the GNSS to maintain location, or else calculate maintain the wearer's 100 current location by employing readings from the accelerometer and/or magnetometer. In some aspects the wearable system may employ GNSS only occasionally in order to maintain the wearer's 100 location. While the airplane is climbing (gaining altitude) or cruising (maintaining steady altitude and speed), the wearable system may resume most functionality, or at least functionality that is allowed by local regulations. In such aspects, the wearable system may enable the cellular connect and reconnect to the Internet. During descent (that is, preparing to land), the wearable system may receive regional rules and disable functionality accordingly. The wearable system may employ GNSS to determine where the wearer 100 is landing, or else if GNSS cannot be employed calculate the wearer's intended destination from accelerometer and magnetometer readings.

During the landing phase of flight (e.g. at latitudes of less than 3,000 m (10,000 feet)), the wearable system may disable certain functionality based on local regulations. Prior to landing, the accelerometer may indicate a steady decrease in altitude, indicating descent. The transition from descent to landing may be indicated by the aircraft reaching an altitude below a given level, or when dropping below a given elevation from takeoff, where elevation is measured as from sea level. The aircraft's touchdown may be indicated by a sharp deceleration, such as from greater tan 160 km/h (100 MPH) to less than 40 km/h (25 mPH), as sensed by the accelerometer. The wearable system may be configured to distinguish touchdown from some other event, such as fall by the wearer 100. Once the wearable system has detected touchdown, it may exit airplane mode and enable all functionality.

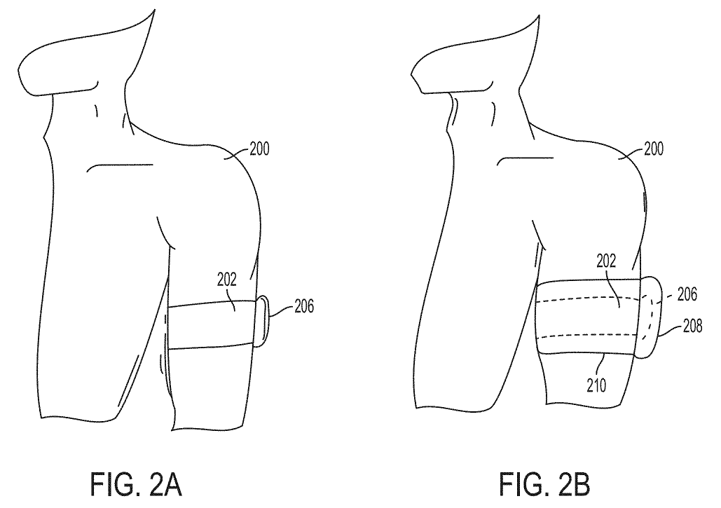

FIG. 2A-2B illustrates an embodiment of a wearable charging station 208 for the wearable system. A wearable charging station 208 allows the batteries of the electronics module to be recharged without having to remove the wearable system. FIG. 2A illustrates a wearable component 202 configured as an armband, and an electronics module 206 configured to be removably attached to the armband. FIG. 2B illustrates the wearable system with a wearable charging station 208. The wearable charging station 208 is configured to partially or fully enclose the electronics module 206. The wearable charging station 208 comprises a strap 210 for attaching the wearable charging station 208 to the wearer 200. The wearable charging station 208 may charge the electronics module by induction. For example, the wearable charging station 208 may comprise an induction coil. The wearable charging station 208 may further comprise a port, such as an AC/DC plug or USB port, for attaching a source of power. The wearable charging station 208 may further removable and/or rechargeable batteries, and thus be operable to provide charge to the electronics module 206 with or without an external source of power. In some aspects, the wearable charging station 208 initiates charging of the electronics module 206 by press of a button on the wearable charging station 208. In some aspects, the charging station 208 initiates charging automatically upon being attached to the electronics module 206.

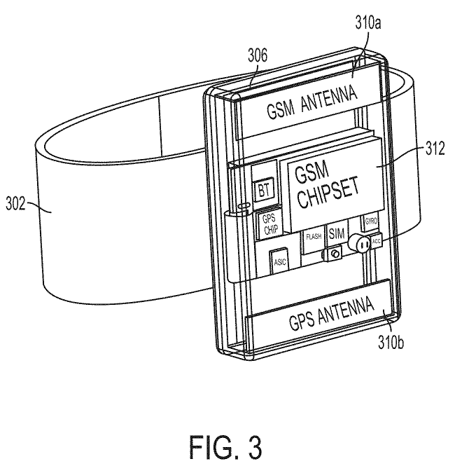

FIG. 3 illustrates a cutaway view of the electronic module 306 of one embodiment of the wearable system. FIG. 3 illustrates the wearable system configured as an armband 302 and an electronics module 306 configured to be removably attached to the armband. In some aspects, the electronics module 306 comprises one or more antenna 310a-310b. The one or more antenna 310a-310b may comprise a GPS antenna, a Global System for Mobile (GSM) antenna, a radio antenna, and/or any other antenna-driven communications interface. The electronics module 306 may further comprise a circuit 312 as necessary to implement the functionality described above.

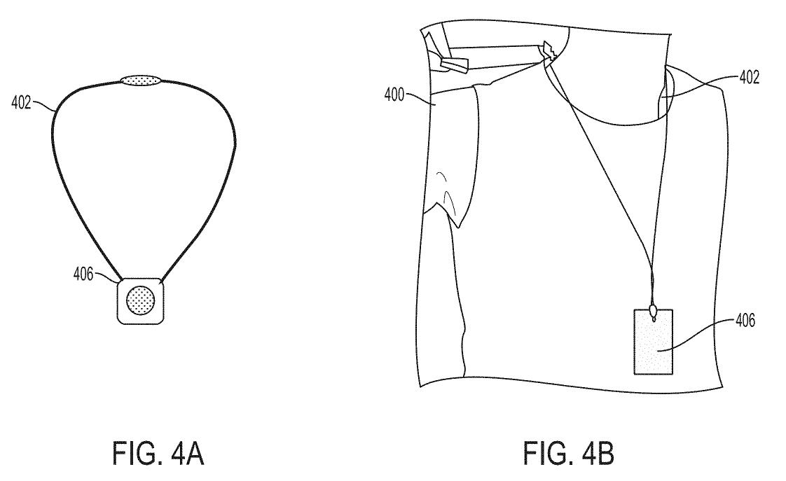

FIGS. 4A-4B illustrate one embodiment of the wearable system configured as a necklace 402 and pendant 406. FIG. 4A illustrates an electronics module configured as a pendant 406. The pendant 406 may be removably or permanently attached to the necklace 402. The pendant 406 is attached to a necklace 402 such that the wearable system can be worn around the neck of the wearer 400. FIG. 4B illustrates the wearable system as worn by the wearer 400. The pendant 406 may rest against the torso of the wearer 400 in order to make an electrical contact with the wearer 400. In some aspects, the necklace 402 strap may be configured to provide an electrical contact with the wearer 400.

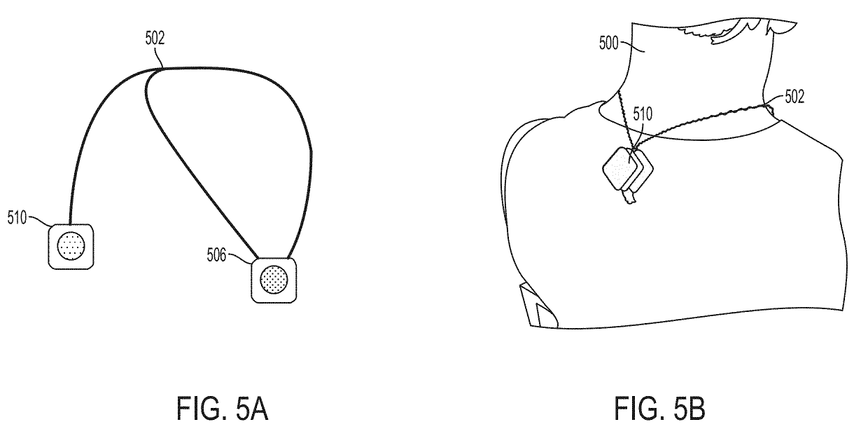

FIGS. 5A-5B illustrate one embodiment of the wearable system configured as a pendant 506 and counterweight 510. FIG. 5A illustrates an electronics module configured as a pendant 506. The pendant 506 is attached to a necklace 502 such that the wearable system can be worn around the neck of the wearer 500. The wearable system further comprises a second electronics module configured as a counterweight 510. The counterweight 510 functions to counterbalance the weight of the pendant 506. FIG. 5B illustrates the wearable system as worn by the wearer 500. The counterweight 510 rests on the back of the wearer 500 while the pendant 506 rests on the wearer's 500 chest or torso. The pendant 506 and/or the counterweight 510 may establish an electrical connection with the body of the wearer 500. In some aspects, the pendant 506 comprises a first electrode and the counterweight 510 comprises a second electrode to form a dipole.

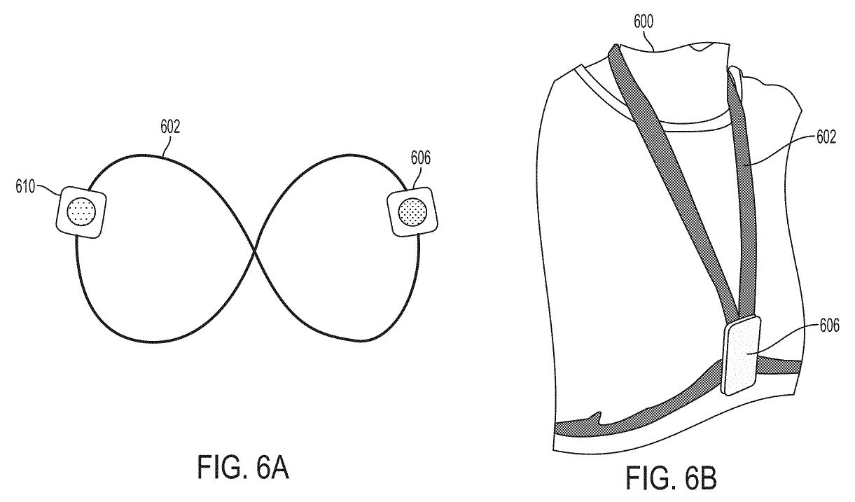

FIGS. 6A-6B illustrate one embodiment of the wearable system configured as a shoulder strap 602. FIG. 6A illustrates a shoulder strap 602 configured approximately in a figure-eight, with an electronics module 606 attached at a first end. A second electronics module 606 may be attached to a second end of the shoulder strap 602. Either or both electronics modules 606, 610 maybe removably or permanently attached to the shoulder strap 602. FIG. 6B illustrates the wearable system as worn by the wearer 600. The electronics module 606 may rest on the chest of the wearer 600 and the second electronics module 610 may rest on the back of the wearer 600. The electronics modules 606, 610 may establish electrical connections with the body of the wearer 600.

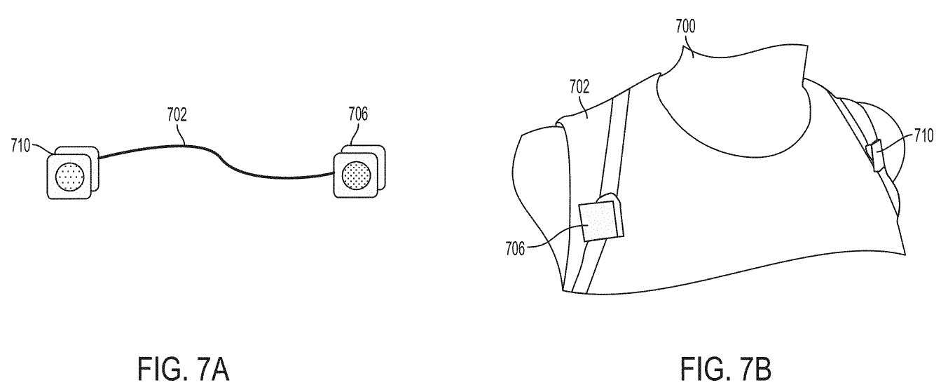

FIGS. 7A-7B illustrate one embodiment of the wearable system configured as a tether 702. FIG. 7A illustrates a tether 702 connected at a first end to an electronics module 706. A second electronics module 710 may be attached to the second end of the tether 702. Either or both electronics modules may be removably or permanently attached to the tether 702. FIG. 7B illustrates one embodiment of the tether 702 as worn by a wearer 700. In the illustrated embodiment, the tether 702 is worn around the back of the neck of the wearer 700, and the electronics module 706 rests on the wearer's 700 shoulder joint. The second electronics module 710 may rest on the wearer's 700 other shoulder joint. The electronics modules 706, 710 may establish an electrical connection with the body of the wearer 700. The electronics modules 706, 710 may rest on the wearer's 700 body and/or be clipped to clothing. In some aspects, the tether 702 may be worn in other configurations.

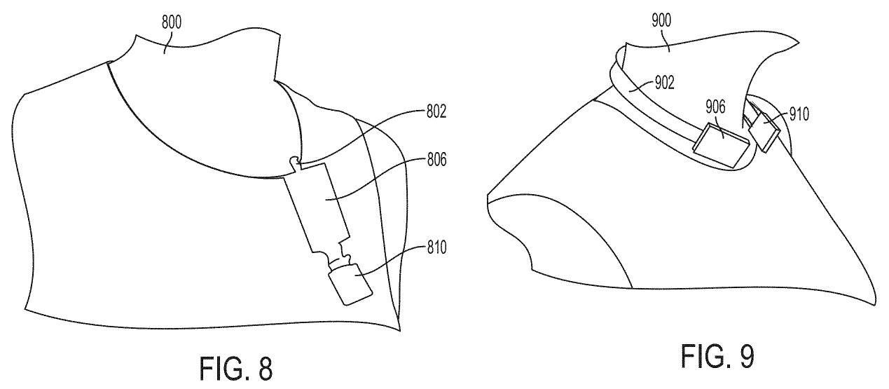

FIG. 8 illustrates one embodiment of a wearable system configured as a clip 802. The clip 802 may be attached to the clothing of the wearer 800. The clip 802 comprises an electronics module 806 that may establish an electrical connection with the body of the wearer 800. The clip 802 may further comprise a second electronics module 810 that may also establish an electrical connection with the body of the wearer 800.

FIG. 9 illustrates one embodiment of a wearable system configured as a neckband 902. The neckband 902 is worn around the neck of the wearer 900. The neckband 902 comprises an electronics module 906 that may establish an electrical connection with the body of the wearer 900. The neckband 902 may further comprise a second electronics module 910 that may also establish an electrical connection with the body of the wearer 900.

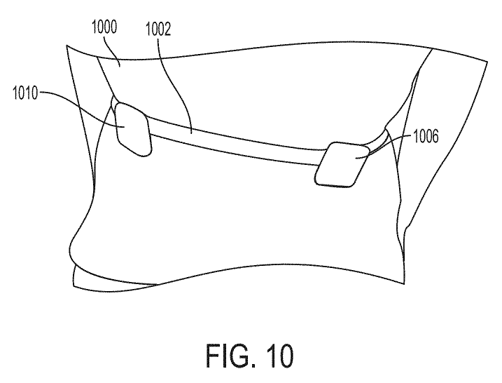

FIG. 10 illustrates one embodiment of a wearable system configured as a waistband 1002. The waistband 1002 is worn around the waist of the wearer 1000. The waistband 1002 comprises an electronics module 1006 that may establish an electrical connection with the body of the wearer. The waistband 1002 may further comprise a second electronics module 1010 that may also establish an electrical connection with the body of the wearer 1000. Either or both electronics modules 1006 and 1010 may be removably or permanently attached to the waistband 1002. The waistband 1002 may also be configured as a chest strap.

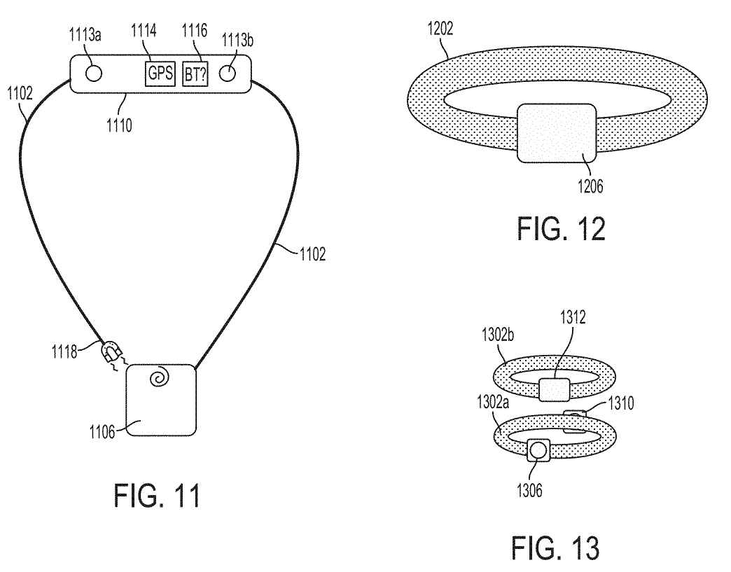

FIG. 11 illustrates one embodiment of a wearable system configured as a necklace 1102 comprising an electronics module configured as a pendant 1106. The pendant 1006 may rest on the chest or torso of the wearer (not shown). The necklace 1102 further comprises a second electronics module 1110. The second electronics module 1110 may rest on the back of the wearer's neck. The second electronics module may comprise one or more electrodes 1113a-1113b. The electrodes 1113a-1113b may contact the back of the wearer's neck in order to establish an electrical connection with the body of the wearer. The second electronics module 1110 may further comprise one or more additional modules 1114, such as communications modules, and/or user interface modules; for example the second electronics module 1110 may comprise a vibration motor for sending alerts to the wearer. In some aspects, the second electronics module 1110 establishes a more stable position than the pendant 1106. In such aspects, the second electronics module 1110 may comprise sensor to detect information about the wearer, such as a fall sensor. The second electronics module 1110 may further comprise a battery 1116. The necklace 1102 may be configured with a plug 1118 that establishes and electrical connection with the pendant 1106. The electrical connection may enable the second electronics module 1110 to provide power to the pendant 1106. The electrical connection may also provide other functionality, such as turning the pendant 1106 on. In some aspects, the pendant 1106 and the second electronics module 1110 may communicate with each other using a wireless protocol, such as Bluetooth (™), instead of or in addition to wired communication.

FIG. 12 illustrates one embodiment of a wearable system configured as a waist or chest band 1202. The waist or chest band comprises an electronics module 1206 that may be removable or permanently attached. In some aspects, the electronics module 1206 comprises all the necessary electronics, communications modules, and/or wearer interface modules. In some aspects, the wearable system makes an electrical contact with the body of the wearer (not shown).

FIG. 13 illustrates one embodiment of a wearable system configured as one or two armbands 1302a, 1302b. The first armband 1302a may have attached thereto a first electronics module 1306 and a second electronics module 1310, operable to provide two electrodes forming a dipole, and/or any of the functionality described above. The wearer may optionally be provided with a second armband 1302b. An third electronics module 1312 attached to the second armband 1302b may provide any or all of the functionality described above, such that some functionality is relocated from the first and second electronics modules 1306, 1310. Alternatively or additionally, the second armband 1302b may be provided alone.

It is understood that other configurations of the wearable system are possible. In some embodiments, the wearable system at least comprises an elastic band capable of drawing one or two electronic modules against the body of the wearer. In such embodiments, the band can be used to provide other functionality. In some embodiments, the wearable system comprises two electronics modules that are not connected. In such embodiments, the electronics modules may communicate with each other with a wireless protocol, such as Bluetooth (™). In some embodiments, the wearable system may be configured as a Bluetooth (™) headset that is placed around the back of the ear; the back of the ear is a relatively stable position on the body. In some embodiments, the wearable system is configures as an article that is worn in pairs, such as earrings, wherein each member of the pair comprises an electrode to establish a dipole. In some embodiments, the wearable system may comprise any wearable article that can be worn close to the body, such as a sports or safety helmet, garter, or garter with a holster. In some embodiments, the wearable system may be incorporated into an article of clothing that is worn close to the body, such as a sports bra or bicycle jersey; in a bicycle jersey, each sleeve cuff may comprise an electrode to form a dipole, and the body of the jersey may be made of a conductive fabric in order for the electrodes to communicate with each other and an electronics module.

### Ingestible Device Indicator System

In various aspects, the wearable system describe above is configured to detect an electrical current signature produced by an ingestible device, such as an ingestible device event indicator. The wearable system may be operable to record and/or transmit the detection of the electrical current signature.

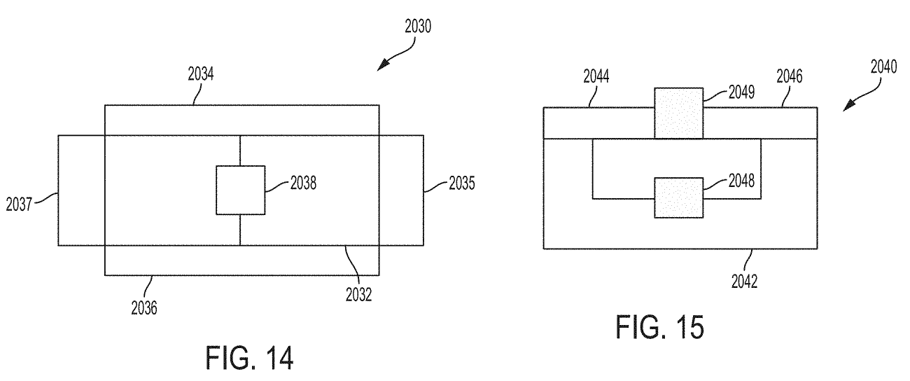

With reference to FIG. 14, there is shown one aspect of an ingestible device event indicator (e.g. ingestible event marker or IEM) system 2030 with dissimilar metals positioned on opposite ends of a framework 2032. The system 2030 can be used in association with any pharmaceutical product to determine when a patient takes the pharmaceutical product. The scope of such an embodiment is not limited by the environment and the product that is used with the system 2030. For example, the system 2030 may be placed within a capsule and the capsule itself may be placed within a conducting liquid. The capsule would then dissolve over a period of time and release the system 2030 into the conducting liquid. Thus, in one aspect, the capsule would contain the system 2030 and no product. Such a capsule may then be used in any environment where a conducting liquid is present and with any product. For example, the capsule may be dropped into a container filled with jet fuel, salt water, tomato sauce, motor oil, or any similar product. Additionally, the capsule containing the system 2030 may be ingested by a living subject at the same time that any pharmaceutical product is ingested in order to record the occurrence of the event, such as when the pharmaceutical product was taken.

In a specific example of the ingestible device event indicator system 2030 combined with a pharmaceutical product, as the product or pill is ingested, the system 2030 is activated. The system 2030 controls conductance to produce a unique current signature that is detected, thereby signifying that the pharmaceutical product has been taken. The system 2030 includes a framework 2032. The framework 2032 is a chassis for the system 2030 and multiple components are attached to, deposited upon, or secured to the framework 2032. Even though the shape of the system 2032 is shown as rectangular, the shape maybe any geometrically suitable shape. In one aspect of the system 2030, a digestible first material 2034 is physically associated with the framework 2032. The first material 2034 may be chemically deposited on, evaporated onto, secured to, or built-up on the framework 2032, all of which may be referred to herein as “deposit” with respect to the framework 2032. The first material 2034 may be deposited by physical vapor deposition, electrodeposition, or plasma deposition, among other protocols. In the illustrated embodiment the first material 2034 is deposited on one side of the framework 2032. The materials of interest that can be used as the first material 2034 include, but are not limited to: Cu or Cul. The first material 2034 may be from about 0.05 to about 500 μm thick, such as from about 5 to about 100 μm thick. The shape may be controlled by shadow mask deposition, or photolithography and etching. Additionally, even though only one region is shown for depositing the first material 2034, each system 2030 may contain two or more electrically unique regions where the first material 2034 may be deposited, as desired.

At a different side of the framework 2032, illustrated in FIG. 7 as the side opposite to the side on which the first material 2034 is located, a digestible second material 2036 is deposited, such that first material 2034 and the second material 2036 are dissimilar. Although not shown, the different side selected may be the side next to the side selected for the first material 2034. The scope of the present disclosure is not limited by the side selected and the term “different side” can mean any of the multiple sides that are different from the first selected side. The materials of interest for the second material 2036 include, but are not limited to: Mg, Zn, or other electronegative metals. As indicated above with respect to the first material 2034, the second material 2036 may be chemically deposited on, evaporated onto, secured to, or built-up on the framework. Additionally, an adhesion layer may be necessary to help the second material 2036 (as well as the first material 2034, when needed) to adhere to the framework 2032. Typical adhesion layers for the second material 2036 are Ti, TiW, Cr or similar material. The second material 2036 and the adhesion layer may be deposited by physical vapor deposition, electrodeposition or plasma deposition. The second material 2036 may be from about 0.05 to about 500 μm thick, such as from about 5 to about 100 μm thick. However, the scope of the present disclosure is not limited by the thickness of any of the materials nor by the type of process used to deposit or secure the materials to the framework 2032.

The first material 2034 and the second material 2036 are selected such that they produce a voltage potential difference when the system 2030 is in contact with conducting liquid, such as for instance body fluids. In such embodiments, one of the first material 2034 or the second material 2036 acts as an anode, while the other of the materials 2034, 2036 acts as a cathode. Thus when the system 2030 is in contact with the conducting liquid, a current path is formed through the conducting liquid between the first material 2034 and the second material 2036. A control device 2038 is secured to the framework 2032 and electrically coupled to the first material 2034 and the second material 2036. The control device 2038 includes electronic circuitry, for example control logic that is capable of controlling and altering the conductance between the materials 2034, 2036.

The voltage potential created between the first material 2034 and the second material 2036 provides the power for operating the system 2030 as well as producing the current flow through the conducting fluid and the system 2030. In one aspect, the system 2030 operates in direct current mode. In an alternative aspect, the system 2030 controls the direction of the current so that the direction of current is reversed in a cyclic manner, similar to alternating current. As the system 2030 reaches the conducting fluid or the electrolyte, where the fluid or electrolyte component is provided by a physiologic fluid, e.g., stomach acid, intestinal fluid, or the like, the path for current flow between the first material 2034 and the second material 2036 is completed external to the system 2030; the current path through the system 2030 is controlled by the control device 2038. Completion of the current path allows for the current to flow and in turn a receiver, not shown, can detect the presence of the current and recognize that the system 2030 has been activated and the desired event is occurring or has occurred.

In one aspect, the two materials 2034, 2036 are similar in function to the two electrodes needed for a direct current power source, such as a battery. The conducting liquid acts as the electrolyte needed to complete the power source. The completed power source described is defined by the physical chemical reaction between the first material 2034 and the second material 2036 of the system 2030 and the surrounding fluids of the body. The completed power source may be viewed as a power source that exploits reverse electrolysis in an ionic or a conductive solution such as gastric fluid, blood, or other bodily fluids and some tissues. Additionally, the environment may be something other than a body and the liquid may be any conducting liquid. For example, the conducting fluid may be salt water or a metallic based paint.

In certain aspects, these two materials 2034, 2036 are shielded from the surrounding environment by an additional layer of material. Accordingly, when the shielding material is dissolved and the two dissimilar materials 2034, 2036 are exposed to the target site, a voltage potential is generated.

Referring again to FIG. 14, the first material 2034 and the second material 2036 provide the voltage potential to activate the control device 2038. Once the control device 2038 is activated or powered up, the control device 2038 can alter conductance between the materials 2034, 2036 in a unique manner. By altering the conductance between the materials 2034, 2036, the control device 2038 is capable of controlling the magnitude of the current through the conducting liquid that surrounds the system 2030. This produces a unique current signature that can be detected and measured by a receiver (not shown), which can be positioned internal or external to the body. In addition to controlling the magnitude of the current path between the materials, non-conducting materials, one or more membrane, or one or more skirts may be used to increase the length of the current path and, hence, act to boost the conductance path, as disclosed in the U.S. patent application Ser. No. 12/238,345 entitled, “In-Body Device with Virtual Dipole Signal Amplification” filed Sep. 25, 2008, the entire content of which is incorporated herein by reference. Alternatively, throughout the disclosure herein, the terms “non-conducting material”, “membrane”, and “skirt” are interchangeable with the term “current path extender” without impacting the scope or the present aspects and the claims herein. A first skirt 2035 and a second skirt 2037 may be associated with, e.g., secured to, the framework 2032. Various shapes and configurations for a skirt 2035, 2037 are contemplated as within the scope of the disclosed embodiments. For example, the system 2030 may be surrounded entirely or partially by a skirt and the skirt maybe positioned along a central axis of the system 2030 or off-center relative to a central axis. Thus, the scope of the present disclosure as claimed herein is not limited by the shape or size of the skirt. Furthermore, in other aspects, the first material 2034 and the second material 2036 may be separated by one skirt that is positioned in any defined region between the materials 2034, 2036.

Referring now to FIG. 15, illustrated is another aspect of an ingestible device indicator system 2040. The system 2040 includes a framework 2042. The framework 2042 is similar to the framework 2032 of FIG. 14. In this aspect of the system 2040, a digestible or dissolvable first material 2044 is deposited on a portion of one side of the framework 2042. At a different portion of the same side of the framework 2042, a digestible second material 2046 is deposited, such that the first material 2044 and the second material 2046 are dissimilar. More specifically, the first material 2044 and the second material 2046 are selected such that they form a voltage potential difference when in contact with a conducting liquid, such as body fluids. Thus, when the system 2040 is in contact with and/or partially in contact with the conducting liquid, a current path is formed through the conducting liquid between the first material 2044 and the second material 2046. A control device 2048 is secured to the framework 2042 and electrically coupled to the first material 2044 and the second material 2046. The control device 2048 includes electronic circuitry that is capable of controlling at least part of the conductance path between the materials 2044, 2046. The materials 2044, 2046 are separated by a non-conducting skirt 2049. Various examples of the skirt 2049 are disclosed in U.S. Provisional Application No. 61/173,511 filed on Apr. 28, 2009 and entitled “HIGHLY RELIABLE INGESTIBLE EVENT MARKERS AND METHODS OF USING SAME” and U.S. Provisional Application No. 61/173,564 filed on Apr. 28, 2009 and entitled “INGESTIBLE EVENT MARKERS HAVING SIGNAL AMPLIFIERS THAT COMPRISE AN ACTIVE AGENT”; as well as U.S. application Ser. No. 12/238,345 filed Sep. 25, 2008 and published as 2009/0082645, entitled “IN-BODY DEVICE WITH VIRTUAL DIPOLE SIGNAL AMPLIFICATION”; the entire disclosure of each is incorporated herein by reference.

Once the control device 2048 is activated or powered up, the control device 2048 can alter conductance between the first material 2044 and the second material 2046. Thus, the control device 2048 is capable of controlling the magnitude of the current through the conducting liquid that surrounds the system 2040. As indicated above with respect to system 2030, a unique current signature that is associated with the system 2040 can be detected by a receiver (not shown) to mark the activation of the system 2040. In order to increase the length of the current path the size of the skirt 2049 may be altered. The longer the current path, the easier it may be for the receiver to detect the current.

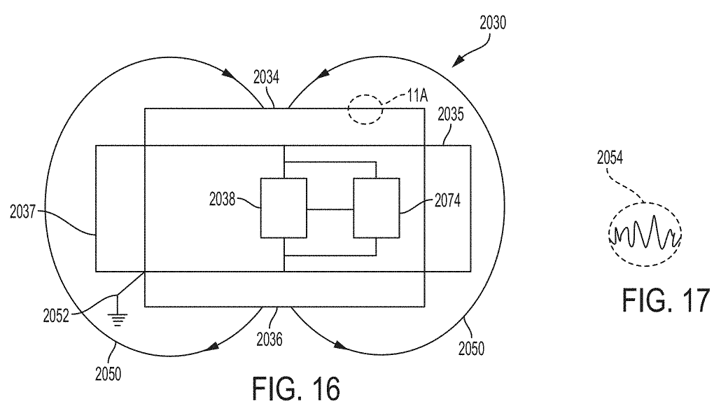

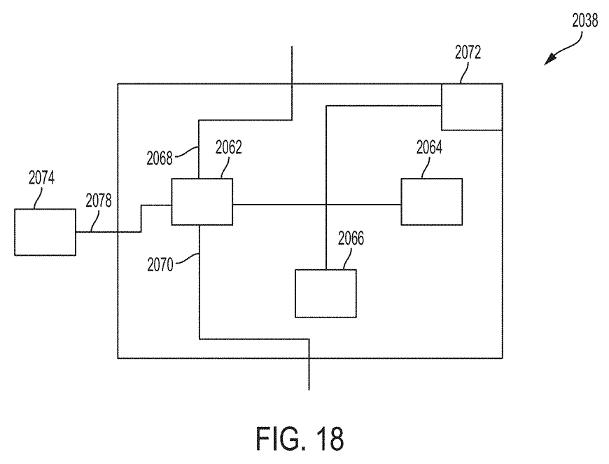

Referring now to FIG. 16, the system 2030 of FIG. 14 is shown in an activated state and in contact with conducting liquid. The system 2030 is grounded through ground contact 2052. Ion or current paths 2050 form between the first material 2034 and the second material 2036 through the conducting fluid in contact with the system 2030. The voltage potential created between the first material 2034 and the second material 2036 is created through chemical reactions between materials 2034, 2036 and the conducting fluid. The system 2030 also includes a first sensor module 2074, which is described in greater detail with respect to FIG. 18.

FIG. 17 shows an exploded view of the surface of the first material 2034. The surface of the material 2034 is not planar, but rather an irregular surface 2054 as shown. The irregular surface 2054 increases the surface area of the material and, hence, the area that comes in contact with the conducting fluid. FIG. 17 illustrates the surface of the first material 2034 by way of example only. It is understood that the second material 2036 may have a similar surface.

In one aspect, at the surface of the first material 2034, there is chemical reaction between the first material 2034 and the surrounding conducting fluid such that mass is released into the conducting fluid. The term “mass” as used herein refers to protons and neutrons that form a substance. One example includes the instant where the material is CuCI and when in contact with the conducting fluid, CuCI becomes Cu (solid) and Cl— in solution. The flow of ions into the conduction fluid is depicted by the ion paths 2050, illustrated in FIG. 16. In a similar manner, there is a chemical reaction between the second material 2036 and the surrounding conducting fluid such that ions are captured by the second material 2036. The release of ions at the first material 2034 and capture of ions by the second material 2036 is collectively referred to as the ionic exchange. The rate of ionic exchange and, hence the ionic emission rate or flow, is controlled by the control device 2038. The control device 2038 can increase or decrease the rate of ion flow by altering the conductance, which alters the impedance, between the materials 2034, 2036. Through controlling the ion exchange, the system 2030 can encode information in the ionic exchange process. Thus, the system 2030 uses ionic emission to encode information in the ionic exchange.

The control device 2038 can vary the duration of a fixed ionic exchange rate or current flow magnitude while keeping the rate or magnitude near constant, similar to when the frequency is modulated and the amplitude is constant. Also, the control device 2038 can vary the level of the ionic exchange rate or the magnitude of the current flow while keeping the duration near constant. Thus, using various combinations of changes in duration and altering the rate or magnitude, the control device 2038 encodes information in the current flow or the ionic exchange. For example, the control device 2038 may use, but is not limited to any of the following techniques namely, Binary Phase-Shift Keying (BPSK or PSK), Frequency modulation, Amplitude modulation, on-off keying, and PSK with on-off keying.

As indicated above, the various aspects disclosed herein, such as the systems 2030, 2040 of FIGS. 14 and 15, include electronic components as part of the control device 2038, 2048. Components that may be present include but are not limited to: logic and/or memory elements, an integrated circuit, an inductor, a resistor, and sensors for measuring various parameters. Each component may be secured to the framework and/or to another component. The components on the surface of the support may be laid out in any convenient configuration. Where two or more components are present on the surface of the solid support, interconnects may be provided.

As indicated above, the system, such as the systems 2030, 2040 of FIGS. 14 and 15, control the conductance between the dissimilar materials 2034, 2036, 2044, 2046 and, hence, the rate of ionic exchange or the current flow. Through altering the conductance in a specific manner the system is capable of encoding information in the ionic exchange and the current signature. The ionic exchange or the current signature is used to uniquely identify the specific system 2030, 2040. Additionally, the systems 2030, 2040 are capable of producing various different unique exchanges or signatures and, thus, provide additional information. For example, a second current signature based on a second conductance alteration pattern may be used to provide additional information, which information may be related to the physical environment. To further illustrate, a first current signature may be a very low current state that maintains an oscillator on the chip and a second current signature may be a current state at least a factor of ten higher than the current state associated with the first current signature.

Referring now to FIG. 18, a block diagram representation of the control device 2038 is shown. The control device 2030 includes a control module 2062, a counter or clock 2064, and a memory 2066. Additionally, the device 2038 is shown to include a second sensor module 2072. The control module 2062 is also in communication with the first sensor module 2074, which was referenced in FIG. 16. Referring again to FIG. 18, the control module 2062 has an input 2068 electrically coupled to the first material 2034 and an output 2070 electrically coupled to the second material 2036. The control module 2062, the clock 2064, the memory 2066, and the sensor modules 2072, 2074 also have power inputs (some not shown). The power for each of these components is supplied by the voltage potential produced by the chemical reaction between first material 2034 and the second material 2036 and the conducting fluid, when the system 2030 is in contact with the conducting fluid. The control module 2062 controls the conductance through logic that alters the overall impedance of the system 2030. The control module 2062 is electrically coupled to the clock 2064. The clock 2064 provides a clock cycle to the control module 2062. Based upon the programmed characteristics of the control module 2062, when a set number of clock cycles have passed, the control module 2062 alters the conductance characteristics between materials 2034, 2036. This cycle is repeated and thereby the control device 2038 produces a unique current signature characteristic. The control module 2062 is also electrically coupled to the memory 2066. Both the clock 2064 and the memory 2066 are powered by the voltage potential created between the first material 2034 and the second material 2036.

The control module 2062 is also electrically coupled to and in communication with the sensor modules 2072, 2074. In the aspect shown, the second sensor module 2072 is part of the control device 2038 and the first sensor module 2074 is a separate component. In alternative aspects, either one of the sensor modules 2072, 2074 can be used without the other. Furthermore, the scope of the present disclosure is not limited by the structural or functional location of the sensor modules 2072, 2074. Additionally, any component of the system 2030 may be functionally or structurally moved, combined, or repositioned without limiting the scope of the present disclosure as claimed. Thus, it is possible to have one single structure, for example a processor, which is designed to perform the functions of all of the following modules: the control module 2062, the clock 2064, the memory 2066, and the first sensor module 2072, and/or the second sensor module 2074. On the other hand, it is also within the scope of the present disclosure to have each of these functional components located in independent structures that are linked electrically and able to communicate. In another aspect, not shown, the clock 2064 and the memory 2066 can be combined into one device.

Referring again to FIG. 18, the sensor modules 2072, 2074 can include any of the following sensors: temperature, pressure, pH level, and/or conductivity. In one aspect, the sensor modules 2072, 2074 gather information from the environment and communicate the analog information to the control module 2062. The control module then converts the analog information to digital information and the digital information is encoded in the current flow or the rate of the transfer of mass that produces the ionic flow. In another aspect, the sensor modules 2072, 2074 gather information from the environment and convert the analog information to digital information and then communicate the digital information to control module 2062. In the aspect shown in FIG. 16, the first sensor module 2074 is shown as being electrically coupled to the first material 2034 and the second material 2036 as well as the control device 2038. In another aspect, as shown in FIG. 18, the first sensor module 2074 is electrically coupled to the control device 2038 at a connection 2078. The connection 2078 acts as both a source for power supply to the sensor module 2074 and a communication channel between the first sensor module 2074 and the control device 2038.

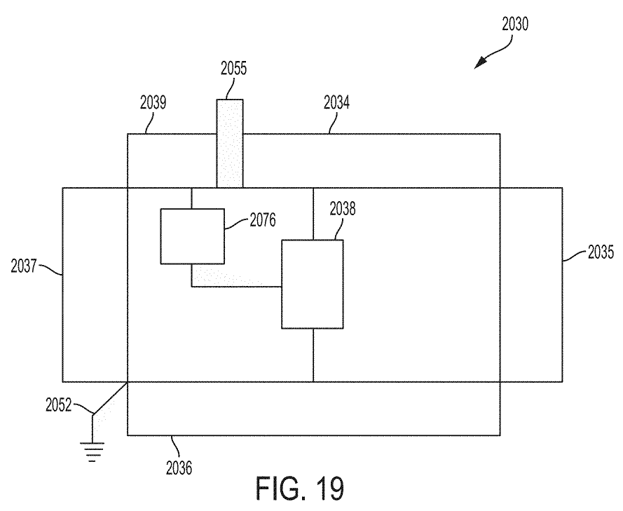

Referring now to FIG. 19, in some aspects the system 2030 includes a pH sensor module 2076 connected to a material 2039, where the material 2039 is selected in accordance with the specific type of sensing function being performed. The pH sensor module 2076 is also connected to the control device 2038. The material 2039 is electrically isolated from the first material 2034 by a non-conductive barrier 2055. In one aspect, the material 2039 is platinum. In operation, the pH sensor module 2076 uses the voltage potential difference between the first material 2034 and the second material 2036. The pH sensor module 2076 measures the voltage potential difference between the first material 2034 and the material 2039 and records that value for later comparison. The pH sensor module 2076 also measures the voltage potential difference between the material 2039 and the second material 2036 and records that value for later comparison. The pH sensor module 2076 calculates the pH level of the surrounding environment using the voltage potential values. The pH sensor module 2076 provides that information to the control device 2038. The control device 2038 varies the rate of the transfer of mass that produces the ionic transfer and the current flow to encode the information relevant to the pH level in the ionic transfer, which can be detected by a receiver (not shown). Thus, the system 2030 can determine and provide the information related to the pH level to a source external to the environment.

As indicated above, the control device 2038 can be programmed in advance to output a pre-defined current signature. In another aspect, the system 2030 can include a receiver system that can receive programming information when the system 2030 is activated.

In addition to the above components, the system 2030 may also include one or other electronic components. Electrical components of interest include, but are not limited to: additional logic and/or memory elements, e.g., in the form of an integrated circuit; a power regulation device, e.g., battery, fuel cell or capacitor; a sensor, a stimulator, etc.; a signal transmission element, e.g., in the form of an antenna, electrode, coil, etc.; a passive element, e.g., an inductor, resistor, etc.

### Receiver

The wearable system described above is also referred to herein as a personal communication system and body-associated personal communicator.

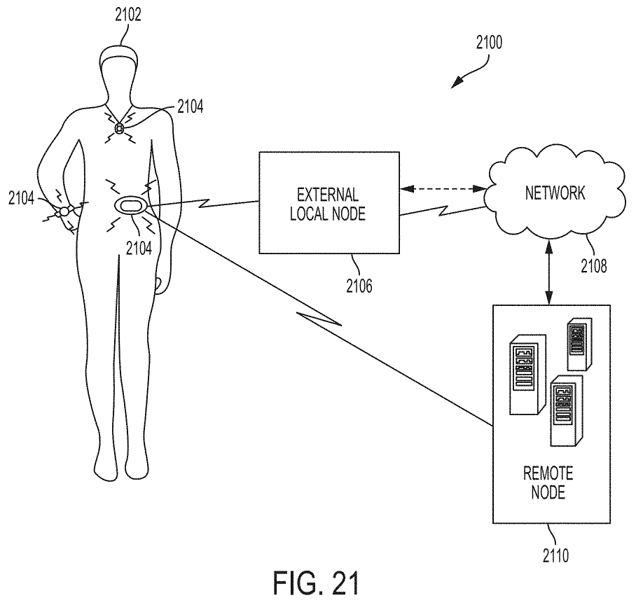

FIG. 21 illustrates one aspect of a personal communication system 2100. As illustrated in FIG. 21, a receiver, otherwise referred to herein as a body-associated personal communicator 2104, is positioned on a living subject 2102. The living subject 2102 may be a human or non-human being. In various aspects, the body-associated personal communicator 2104 may be realized in many forms and configurations including sensor-enabled patches, watches, and jewelry, as shown in FIG. 21, for example, as well as a bandage with an adhesive portion, wristbands, earrings, bracelets, rings, pendants, clothing, undergarments, hats, caps, scarves, pins, accessories, belts, shoes, eyeglasses, contact lenses, hearing-aides, subcutaneous implants, and other devices that are wearable, implantable, or semi-implantable on or in the living subject 2102 without limitation. The body-associated personal communicator 2104 may be configured to communicate with the living subject 2102 and an external local node 2106. The external local node 2106 may be configured to communicate with a remote node 2110 via a network 2108. The remote node 2110 may communicate with the network 2108 using via wired or wireless links 2150. In one aspect, the body-associated personal communicator 2104 is configured to communicate with the remote node 2110 directly 2152. It will be appreciated that in the context of the present disclosure, communication is intended to encompass communications to and from the personal communicator 2104 and the external local node 2106. Likewise, communication is intended to encompass communications to and from the body-associated personal communicator 2104 and the remote node 2110 as well as communications to and from the external local node 2106 and the remote node 2110.

The body-associated personal communicator 2104 may comprise any number of distinct physiologic parameter or biomarker collecting and/or sensing capabilities. The number of distinct parameters or biomarker collecting and/or sensing capabilities may vary e.g., one or more, two or more, three or more, four or more, five or more, ten or more, and so on. In certain configurations, the body-associated personal communicator 2104 comprises one or more active components that are able to dynamically monitor and record individual physiologic parameters and/or biomarkers associated with the living subject 2102. Such components include, without limitation, sensors, electronic recording devices, processors, memory, communication components. In one aspect, the body-associated personal communicator 2104 may include an on-board battery to supply electrical power to the active components. The physiologic parameter or biomarker sensing abilities may include sensing cardio-data, including heart rate, electrocardiogram (ECG), and the like, respiration rate, temperature, pressure, chemical composition of fluid, e.g., analyte in blood, fluid state, blood flow rate, physical activity, sleep, accelerometer motion data, without limitation, for example.

In one aspect, the body-associated personal communicator 2104 provides specific information about the physiologic state of the subject 2102. In another aspect, some of this information may be derived from sensors embedded in the body-associated personal communicator 2104. The subject 2102 may obtain the body-associated personal communicator 2104 with a prescription, for example, and then wear the body-associated personal communicator 2104 for a prescribed period, e.g., hours, days, weeks, months, years.

In one aspect, the body-associated personal communicator 2104 is configured to (a) monitor and record individual physiology, e.g., physical activity, heart rate, respiration, temperature, sleep, fluidics information, etc., of the living subject 2102 and (b) communicate these parameters beyond the body of the living subject 2102 to other client devices, e.g., mobile phones, computers, internet servers, etc., in order to (c) enable support and collaboration for fitness, well-being, disease management, sport, entertainment, gaming, social goals and other applications on a social media platform.

A challenge for such body-associated personal communicators 2104 is creating a compelling rationale for the individual 2102 to wear or use the body-associated personal communicator 2104 on a continuous basis—for example, to apply an adhesive bandage-based body-associated personal communicator 2104 to their skin for weeks, months and potentially years and accept the possibility of its inconveniences and limitations, such as (i) potential skin irritation, (ii) the burden of frequent application and removal, and (iii) a feeling of intrusiveness into the wearer's daily life. An opportunity for the personal communicator 2104 is to exploit fundamental “intimacy” advantages they have over other sensor-enabled and communication devices that are not worn on or in the body. A body-associated personal communicator 2104 interface with the individual 2102 is by definition highly personal and tangible, with the ability to have private, communication between the individual and the personal communicator (leveraging physical, tactile “body language” or other signals), where the communication is substantially undetectable by others. In this manner, the body-associated personal communicator 2104 may enable product and service possibilities not feasible with other approaches. The body language opportunity seeks to overcome at least some of the challenges and burdens of the body-associated personal communicator 2104 to create a compelling rationale to make the body-associated personal communicator 2104 as indispensable to a consumer as the mobile phone as an extension of their mind and body. In one aspect, discreet communications between the body-associated personal communicator 2104 and the living subject 2102 can be auditory via a small earpiece placed inside the ear canal, or visual via images projected on specialized eye glasses worn by living subject 2102. In other aspects, discreet modes of communication between the living subject 2102 and the personal communicator 2104 include, without limitation, visual, auditory, vibratory, tactile, olfactory, and taste as described in the form of illustrative examples hereinbelow.

In one aspect, the body-associated personal communicator 2104, for example a sensor patch that adheres to the skin of an individual such as the living subject 2102, communicates with its wearer by sending and receiving tactile or other signals. The default settings may be modified such that the body-associated personal communicator 2104 discreetly vibrates or pulses in a specific manner or pattern, e.g., time or space based, to remind the subject 2102 of important events or to communicate important personalized messages to the wearer. The default settings also may be modified such that the subject 2102 can transmit and record meaningful inputs and messages to the body-associated personal communicator 2104 by communicating a simple language of finger taps, jiggles, scratches or other physical inputs initiated by the subject 2102. Through the body-associated personal communicator 2104 communications architecture, e.g., a BLUETOOTH or other communication links to other devices beyond the body, the composite set of sensed physiology, tactile inputs, and outputs can be transmitted to other individuals, groups, caregivers, and related products, e.g., online games, of the subject's 2102 choosing via the external local node 2106, network 2108, and/or the remote node 2110. The features of the body-associated personal communicator 2104 are based on a sustained behavior change mechanism and it increases the value and potential of body-associated personal communicators 2104 and the likelihood that consumers will seek out, use, and benefit from such body-associated personal communicators 2104.

In-body communications include any communication of data or information via the body of the living subject 2102, i.e., communication via or associated with inter-body aspects, intra-body aspects, and a combination of the same. For example, inter-body aspects include communications associated with devices designed to attach to a body surface. Intra-body aspects include communications associated with data generated from within the body, e.g., by the body itself or by a device implanted, ingested, or otherwise locatable in, or partially in, the body. For example, intra-body communications are disclosed in the U.S. Provisional Patent Application No. 61/251088, the entire content of which is hereby incorporated by reference. Communications include and/or may be associated with software, hardware, circuitry, various devices, and combinations thereof. The devices include devices associated with physiologic data generation, transmission, reception, communication. The devices further include various implantable, ingestible, insertable, and/or attachable devices associated with the human body or other living organisms. The devices still further include multimedia devices such as telephones, stereos, audio players, PDAs, handheld devices, and multimedia players.

The system for incorporating physiologic data enables exchange, transmission, receipt, manipulation, management, storage, and other activities and events related to physiologic data. Such activities and events may be contained within the system for incorporating physiologic data, partially integrated with the system for incorporating physiologic data, or associated with externalities, e.g., activities, systems, components, and the like which are external to the system for incorporating physiologic data. The physiologic data environment includes any source of information or data, including remote computer systems, local computer devices. The information or data may comprise physiologic data in whole or in part, e.g., aggregated or generated with other types of data. The physiologic data may be pure or refined, e.g., physiologic data from which inferences are drawn.

### External Local Node

As shown in FIG. 21, the body-associated personal communicator 2104, regardless of form factor or implementation, is in communication with an external local node 2106. In one aspect, the body-associated personal communicator 2104 includes the capability of communicating, e.g., receiving, transmitting, generating, and recording data directly or indirectly from the living subject 2102. Although the data may include physiologic data, it is not limited as such. Any data of a physiologic nature may be associated with the living subject 2102. The physiologic data may include, for example, heart rate, heart rate variability, respiration rate, body temperature, temperature of local environment, three-axis measurement of activity and torso angle, as well as other physiologic data, metrics, inertial measurements comprising at least an accelerometer, a gyroscope, and a magnetometer, and indicators associated with one or more individuals. The physiologic data may be communicated at various times or time intervals to the external local node 2106. For example, the communication may be real-time, i.e., in close temporal proximity to a time in which the physiologic data were generated, measured, ascertained, or on an historical basis, i.e., in far temporal proximity to a time in which the physiologic data was generated, measured, ascertained. In various aspects, the physiologic data may be associated with a variety of devices, e.g., cardiac device.

Broad categories of external local nodes 2106 include, for example, base stations, personal communication devices, handheld devices, and mobile telephones. In various aspects, the external local node 2106 may be implemented as a handheld portable device, computer, mobile telephone, sometimes referred to as a smartphone, tablet personal computer (PC), kiosk, desktop computer, laptop computer, game console, or any combination thereof. Although some aspects of the external local node 2106 may be described with a mobile or fixed computing device implemented as a smart phone, personal digital assistant, laptop, desktop computer by way of example, it may be appreciated that the various aspects are not limited in this context. For example, a mobile computing device may comprise, or be implemented as, any type of wireless device, mobile station, or portable computing device with a self-contained power source, e.g., battery, such as the laptop computer, ultra-laptop computer, personal digital assistant (PDA), cellular telephone, combination cellular telephone/PDA, mobile unit, subscriber station, user terminal, portable computer, handheld computer, palmtop computer, wearable computer, media player, pager, messaging device, data communication device, and so forth. A fixed computing device, for example, may be implemented as a desk top computer, workstation, client/server computer, and so forth.

In one aspect, external local node 2106 comprises personal communication devices including, for example, devices having communication and computer functionality and typically intended for individual use, e.g., mobile computers, sometimes referred to as “handheld devices.” Base stations comprise any device or appliance capable of receiving data such as physiologic data. Examples include computers, such as desktop computers and laptop computers, and intelligent devices/appliances. Intelligent devices/appliances include consumer and home devices and appliances that are capable of receipt of data such as physiologic data. Intelligent devices/appliances may also perform other data-related functions, e.g., transmit, display, store, and/or process data. Examples of intelligent devices/appliances include refrigerators, weight scales, toilets, televisions, door frame activity monitors, bedside monitors, bed scales. Such devices and appliances may include additional functionality such as sensing or monitoring various physiologic data, e.g., weight, heart rate. Mobile telephones include telephonic communication devices associated with various mobile technologies, e.g., cellular networks.

In various aspects, the handheld device includes software, e.g., a software agent/application, associated with the physiologic data. In various aspects of the handheld device, the software is preconfigured, i.e., configurable by the manufacturer/retailer; configurable by the consumer, i.e., downloadable from a website; or a combination of the same.

In various aspects, the external local node 2106 may provide voice and/or data communications functionality in accordance with different types of cellular radiotelephone systems. Examples of cellular radiotelephone systems may include Code Division Multiple Access (CDMA) systems, Global System for Mobile Communications (GSM) systems, North American Digital Cellular (NADC) systems, Time Division Multiple Access (TDMA) systems, Extended-TDMA (E-TDMA) systems, Narrowband Advanced Mobile Phone Service (NAMPS) systems, 3G systems such as Wde-band CDMA (WCDMA), CDMA-2000, Universal Mobile Telephone System (UMTS) systems, WiMAX (Worldwide Interoperability for Microwave Access, LTE (Long Term Evolution) and so forth.

In various embodiments, the external local node 2106 may be configured to provide voice and/or data communications functionality in accordance with different types of wireless network systems or protocols. Examples of suitable wireless network systems offering data communication services may include the Institute of Electrical and Electronics Engineers (IEEE) 802.xx series of protocols, such as the IEEE 802.1a/b/g/n series of standard protocols and variants (also referred to as “WFi”), the IEEE 802.16 series of standard protocols and variants (also referred to as “WiMAX”), the IEEE 802.20 series of standard protocols and variants, and so forth. A mobile computing device may also utilize different types of shorter range wireless systems, such as a BLUETOOTH system operating in accordance with the Bluetooth Special Interest Group (SIG) series of protocols, including Bluetooth Specification versions v1.0, v1.1, v1.2, v1.0, v2.0 with Enhanced Data Rate (EDR), as well as one or more Bluetooth Profiles, and so forth. Other examples may include systems using infrared techniques or near-field communication techniques and protocols, such as electromagnetic induction (EMI) techniques. Communication includes any method, act, or vehicle of communication, and/or combinations thereof. For example, communication methods include manual, wired, and wireless. Wireless technologies include radio signals, such as x-rays, ultraviolet light, the visible spectrum, infrared, microwaves, and radio waves, etc. Wireless services include voice and messaging, handheld and other Internet-enabled devices, data networking.

In addition to the standard voice function of a telephone, various aspects of mobile telephones may support many additional services and accessories such as short message service (SMS) for text messaging, email, packet switching for access to the Internet, java gaming, wireless, e.g., short range data/voice communications, infrared, camera with video recorder, and multimedia messaging system (MMS) for sending and receiving photos and video. Some aspects of mobile telephones connect to a cellular network of base stations (cell sites), which is, in turn, interconnected to the public switched telephone network (PSTN) or satellite communications in the case of satellite phones. Various aspects of mobile telephones can connect to the Internet, at least a portion of which can be navigated using the mobile telephones.

In various aspects, the mobile telephone includes software, e.g., a software agent/application, associated with the physiologic data. One example is an auto refill application related to or integrated with an auto refill system to facilitate automated prescription refill functions. In various aspects of the mobile telephone, the software is preconfigured, i.e., configurable by the manufacturer/retailer; configurable by the consumer, i.e., downloadable from a website; or a combination of the same.

The mobile telephone includes, for example, devices such as a short-range, portable electronic device used for mobile voice or data communication over a network of specialized cell site base stations. The mobile telephone is sometimes known as or referred to as “mobile,” “wireless,” “cellular phone,” “cell phone,” or “hand phone (HP).”

In one aspect, the external local node 2106 may be configured as a communication hub and may include any hardware device, software, and/or communications component(s), as well as systems, subsystems, and combinations of the same which generally function to communicate physiologic and non-physiologic data between the personal communicator 2104 and the external local node 2106. Communication of the data includes receiving, storing, manipulating, displaying, processing, and/or transmitting the data to the remote node 2110 via the network 2108. In various aspects, the external local node 2106 also functions to communicate, e.g., receive and transmit, non-physiologic data. Example of non-physiologic data include gaming rules and data generated by a separate cardiac-related device such as an implanted pacemaker and communicated to the hub directly or indirectly, e.g., via the personal communicator 2104.

In one aspect, the external local node 2106, for example, a hub, includes a software application associated with a mobile telephone of a patient. The application and mobile telephone function to receive physiologic data from a receiver, which, in turn, receives the physiologic data directly from an individual or indirectly, e.g., via a device. Examples of devices include cardiac devices and ingestible devices. The hub stores, manipulates, and/or forwards the data, alone or in combination with other data, via the network 2108 to a remote node 2110.

In various aspects, the external local node 2106 (hub) receives, generates, communicates, and/or transmits, physiologic data, alone or in combination with other data, i.e., non-physiologic data such as ingestion information from IEMs or various sources. Communication from the external local node 2106 includes any transmission means or carriers, and combinations thereof, including wireless, wired, radio frequency (RF), conductive, etc. as is known in the art or as may become available in the future.

Further, various aspects of the hub include combinations of devices. One such combination is the body-associated personal communicator 2104 in communication with the handheld device or the mobile telephone. Thus, for example, the body-associated personal communicator 2104 wirelessly transmits physiologic data to the mobile telephone having a receiver and a software agent available thereon. The receiver of the mobile telephone receives the physiologic data. A software agent, e.g., an application, processes the physiologic data and displays various information related to the physiologic data via, for example, a customized graphical user interface (GUI). In various aspects, the software agent generates displays with a predetermined “look and feel,” i.e., recognizable to a user as belonging to a predetermined group of software programs, GUIs, source devices, communities, gaming software, etc.

The base station includes systems, subsystems, devices, and/or components that receive, transmit, and/or relay the physiologic data. In various aspects, the base station communicably interoperates with a receiver such as the body-associated personal communicator 2104 and a communications network 2108 such as the Internet. Examples of base stations are computers, e.g., servers, personal computers, desktop computers, laptop computers, intelligent devices/appliances, etc., as heretofore discussed. In various aspects, the base station may be embodied as an integrated unit or as distributed components, e.g., a desktop computer and a mobile telephone in communication with one another and in communication with a patch receiver and the Internet. In various aspects, the base station includes the functionality to wirelessly receive and/or wirelessly transmit data, e.g., physiologic data received from and transmitted to the body-associated personal communicator 2104 and the Internet. Further, in various aspects, the base station may incorporate and/or be associated with, e.g., communicate with, various devices. Such devices may generate, receive, and/or communicate data, e.g., physiologic data. The devices include, for example, “intelligent” devices such as gaming devices, e.g., electronic slot machines, handheld electronic games, electronic components associated with games and recreational activities.

### Network

Vehicles of communication include the network 2108. In various aspects, the network 2108 comprises local area networks (LAN) as well as wide area networks (WAN) including without limitation Internet, wired channels, wireless channels, communication devices including telephones, computers, wire, radio, optical or other electromagnetic channels, and combinations thereof, including other devices and/or components capable of or associated with communicating data. For example, the communication environments include in-body communications, various devices, and various modes of communications such as wireless communications, wired communications, and combinations of the same. As an example and not by way of limitation, one or more portions of network 2108 may include an ad hoc network, an intranet, an extranet, a virtual private network (VPN), a local area network (LAN), a wireless LAN (WLAN), a wide area network (WAN), a wireless WAN (WWAN), a metropolitan area network (MAN), a portion of the Internet, a portion of the Public Switched Telephone Network (PSTN), a cellular telephone network, or a combination of two or more of these. Network 2108 may include one or more networks 2108.

Wireless communication modes include any mode of communication between points that utilizes, at least in part, wireless technology including various protocols and combinations of protocols associated with wireless transmission, data, and devices. The points include, for example, wireless devices such as wireless headsets, audio and multimedia devices and equipment, such as audio players and multimedia players, telephones, including mobile telephones and cordless telephones, and computers and computer-related devices and components, such as printers.

Wired communication modes include any mode of communication between points that utilizes wired technology including various protocols and combinations of protocols associated with wired transmission, data, and devices. The points include, for example, devices such as audio and multimedia devices and equipment, such as audio players and multimedia players, telephones, including mobile telephones and cordless telephones, and computers and computer-related devices and components, such as printers.

Links 2150 may connect the remote node 2110 to the communication network 2108. This disclosure contemplates any suitable links 2150. In particular embodiments, one or more links 2150 include one or more wireline (such as for example Ethernet, Digital Subscriber Line (DSL), or Data Over Cable Service Interface Specification (DOCSIS)), wireless (such as for example W-Fi or Worldwide Interoperability for Microwave Access (WMAX)), or optical (such as for example Synchronous Optical Network (SONET) or Synchronous Digital Hierarchy (SDH)) links. In particular embodiments, one or more links 2150 each include an ad hoc network, an intranet, an extranet, a VPN, a LAN, a WLAN, a WAN, a WWAN, a MAN, a portion of the Internet, a portion of the PSTN, a cellular technology-based network, a satellite communications technology-based network, another link 2150, or a combination of two or more such links 2150. Links 2150 need not necessarily be the same throughout network environment 101. One or more first links 2150 may differ in one or more respects from one or more second links 2150.

### Remote Node

In one aspect, the remote node 2110 comprises social network systems, commercial systems, healthcare systems, pharmacy systems, university systems, financial transaction systems, web communities, physician systems, family caregiver systems, regulatory agency systems, wholesaler/retailer systems as described in U.S. patent application Ser. No. 12/522,249 titled “INGESTIBLE EVENT MARKER DATA SYSTEM,” the disclosure of which is herein incorporated by reference in its entirety. In other aspects, the remote node 2110 comprises state games, behavioral reflective games, psychological response games, synchronization games, actual progress games, and recreational games as described in PCT Patent Application No. PCT/US09/60713 dated Oct. 14, 2009 titled “METHOD AND SYSTEM FOR INCORPORATING PHYSIOLOGIC DATA IN A GAMING ENVIRONMENT” and published as WO 2010/045385, the disclosure of which is herein incorporated by reference in its entirety.

### Receiver Functionality

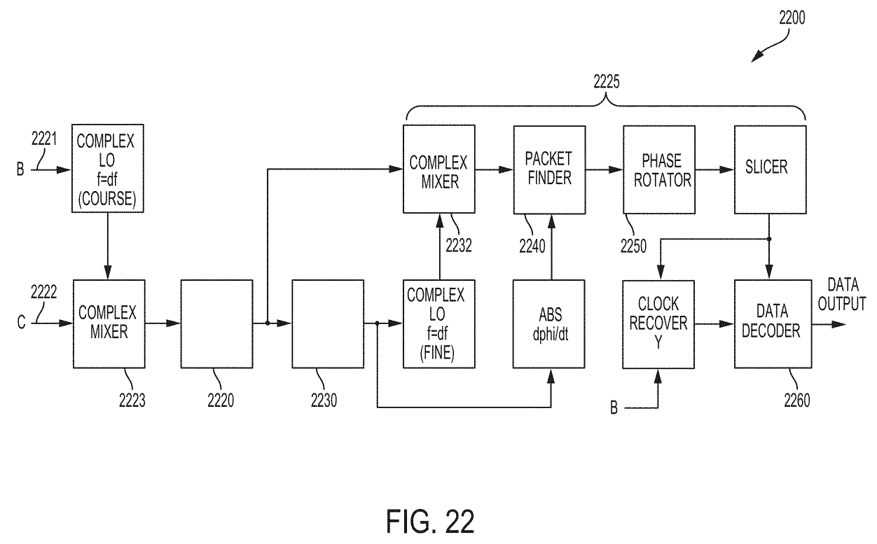

FIG. 22 provides a functional block diagram 2200 of how a receiver (e.g., body-associated personal communicator 2104) may implement a coherent demodulation protocol, according to the disclosed embodiments, in order to read a packet of data present in a signal. It should be noted that only a portion of the receiver is shown in FIG. 22. FIG. 22 illustrates the process of mixing the signal down to baseband once the carrier frequency (and carrier signal mixed down to carrier offset) is determined. A carrier signal 2221 is mixed with a second carrier signal 2222 at a mixer 2223. A narrow low-pass filter 2220 is applied of appropriate bandwidth to reduce the effect of out-of-bound noise. Demodulation occurs at a number of functional blocks 2225 in accordance with the coherent demodulation scheme of the disclosed embodiments. The unwrapped phase 2230 of the complex signal is determined. An optional third mixer stage 2232, in which the phase evolution is used to estimate the frequency differential between the calculated and real carrier frequency, can be applied. The structure of the packet is then leveraged to determine the beginning 2240 of the coding region of the Binary Phase-Shift Keying (BPSK) signal. Mainly, the presence of the sync header, which appears as an FM porch in the amplitude signal of the complex demodulated signal, is used to determine the starting bounds of the packet. Once the starting point of the packet is determined the signal is rotated 2250 on the IQ plane and standard bit identification and eventually decoded 2260.

In addition to demodulation, receiver may include a forward error correction module, which module provides additional gain to combat interference from other unwanted signals and noise. Forward error correction functional modules of interest include those described in PCT Application Serial No. PCT/US2007/024225, the disclosure of which is herein incorporated by reference. In some instances, the forward error correction module may employ any convenient protocol, such as Reed-Solomon, Golay, Hamming, BCH, and Turbo protocols to identify and correct (within bounds) decoding errors.

Receivers of the disclosure, such as the body-associated personal communicator 2104, may further employ a beacon functionality module. In various aspects, the beacon switching module may employ one or more of the following: a beacon wakeup module, a beacon signal module, a wave/frequency module, a multiple frequency module, and a modulated signal module.

The beacon switching module may be associated with beacon communications, e.g., a beacon communication channel, a beacon protocol, etc. For the purpose of the present disclosure, beacons are typically signals sent either as part of a message or to augment a message (sometimes referred to herein as “beacon signals”). The beacons may have well-defined characteristics, such as frequency. Beacons may be detected readily in noisy environments and may be used for a trigger to a sniff circuit, such as described below.

In one aspect, the beacon switching module may comprise the beacon wakeup module, having wakeup functionality. Wakeup functionality generally comprises the functionality to operate in high power modes only during specific times, e.g., short periods for specific purposes, to receive a signal, etc. An important consideration on a receiver portion of a system is that it be of low power. This feature may be advantageous in an implanted receiver, to provide for both small size and to preserve a long-functioning electrical supply from a battery. The beacon switching module enables these advantages by having the receiver operate in a high power mode for very limited periods of time. Short duty cycles of this kind can provide optimal system size and energy draw features.

In practice, the receiver may “wake up” periodically, and at low energy consumption, to perform a “sniff function” via, for example, a sniff circuit. For the purpose of the present application, the term “sniff function” generally refers to a short, low-power function to determine if a transmitter is present. If a transmitter signal is detected by the sniff function, the device may transition to a higher power communication decode mode. If a transmitter signal is not present, the receiver may return, e.g., immediately return, to sleep mode. In this manner, energy is conserved during relatively long periods when a transmitter signal is not present, while high-power capabilities remain available for efficient decode mode operations during the relatively few periods when a transmit signal is present. Several modes, and combination thereof, may be available for operating the sniff circuit. By matching the needs of a particular system to the sniff circuit configuration, an optimized system may be achieved.

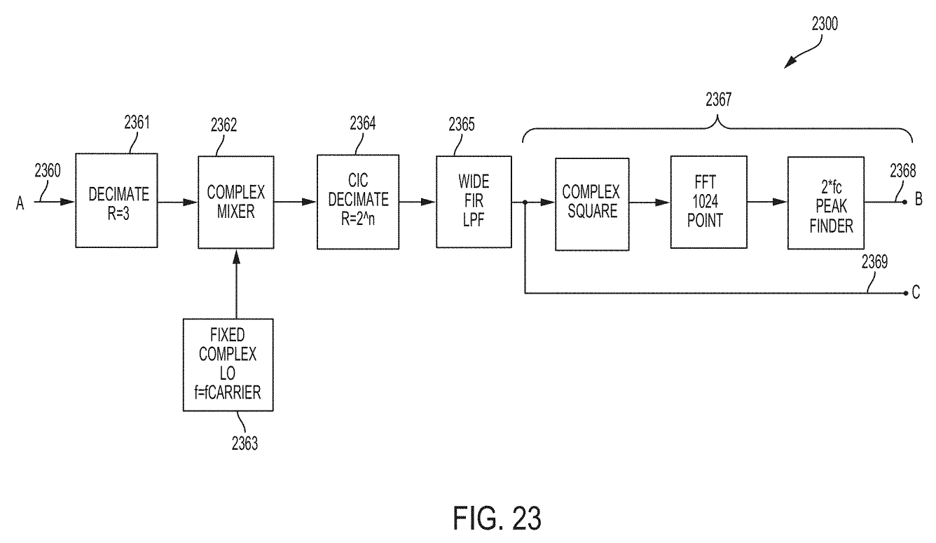

Another view of a beacon module is provided in the functional block diagram shown in FIG. 23. The diagram of FIG. 23 outlines one technique for identifying a valid beacon. The incoming signal 2360 represents the signals received by electrodes, bandpass filtered (such as from 10 KHz to 34 KHz) by a high frequency signaling chain (which encompasses the carrier frequency), and converted from analog to digital. The signal 2360 is then decimated 2361 and mixed at the nominal drive frequency (such as, 12.5 KHz, 20 KHz, etc.) at a mixer 2362. The resulting signal is decimated a second time 2364 and low-pass filtered (such as 5 KHz BW) 2365 to produce the carrier signal mixed down to carrier offset (signal 2369). Signal 2369 is further processed by a series of functions 2367 (fast Fourier transform and then detection of two strongest peaks) to provide the true carrier frequency signal 2368. This protocol allows for accurate determination of the carrier frequency of the transmitted beacon.

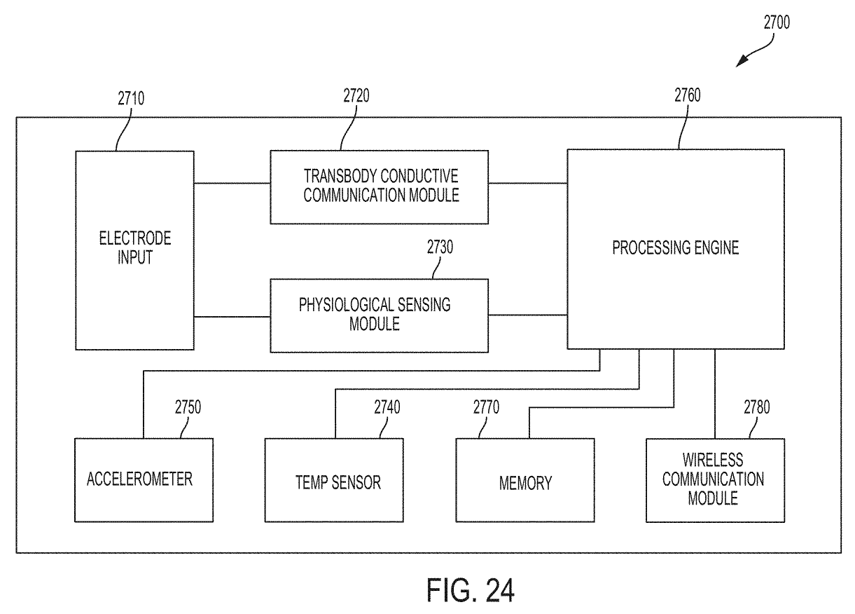

FIG. 24 provides a block functional diagram of an integrated circuit component of a signal receiver (e.g., body-associated personal communicator 2104) according to an aspect of the disclosed embodiments. In FIG. 24, receiver 2700 includes electrode input 2710. Electrically coupled to the electrode input 2710 are a transbody conductive communication module 2720 and physiologic sensing module 2730. In one aspect, transbody conductive communication module 2720 is implemented as a high frequency (HF) signal chain and physiologic sensing module 2730 is implemented as a low frequency (LF) signal chain. Also shown are a CMOS temperature sensing module 2740 (for detecting ambient temperature) and a three-axis accelerometer 2750. Receiver 2700 also includes a processing engine 2760 (for example, a microcontroller and digital signal processor), non-volatile memory 2770 (for data storage) and a wireless communication module 2780 (for data transmission to another device, for example in a data upload action).

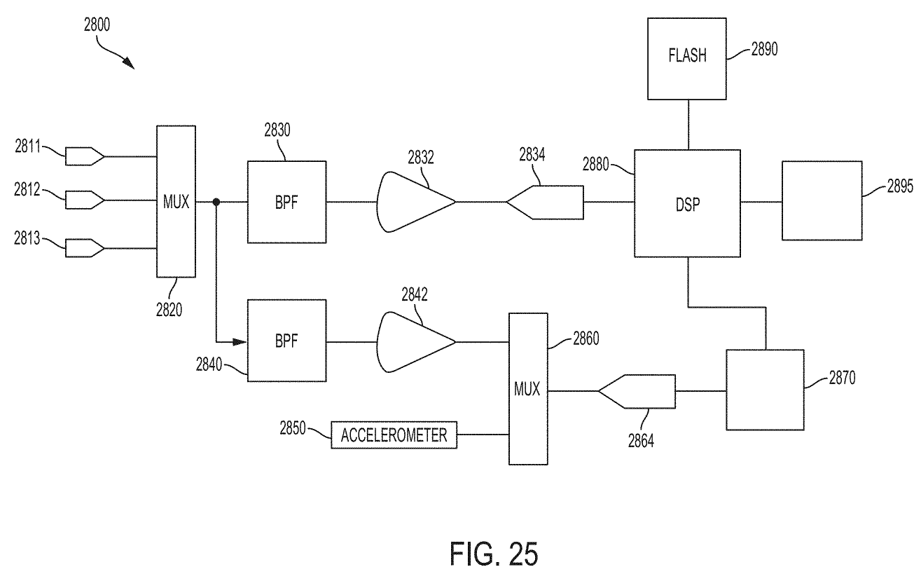

FIG. 25 provides a more detailed block diagram of a circuit configured to implement the block functional diagram of the receiver 2700 (e.g., body-associated personal communicator 2104) depicted in FIG. 24, according to one aspect of the disclosed embodiments. In FIG. 25, receiver 2800 (e.g., body-associated personal communicator 2104) includes electrodes e1, e2 and e3 (2811, 2812 and 2813) which, for example, receive the conductively transmitted signals by an IEM and/or sense physiologic parameters or biomarkers of interest. The signals received by the electrodes 2811, 2812, 2813 are multiplexed by a multiplexer 2820 which is electrically coupled to the electrodes.

The multiplexer 2820 is electrically coupled to both a high band pass filter 2830 and a low band pass filter 2840. The high and low frequency signal chains provide for programmable gain to cover the desired level or range. In this specific aspect, high band pass filter 2830 passes frequencies in the 10 KHz to 34 KHz band while filtering out noise from out-of-band frequencies. This high frequency band may vary, and may include, for example, a range of 3 KHz to 300 KHz. The passing frequencies are then amplified by and amplifier 2832 before being converted into a digital signal by a converter 2834 for input into a high power processor 2880 (shown as a DSP) which is electrically coupled to the high frequency signal chain.

The low band pass filter 2840 is shown passing lower frequencies in the range of 0.5 Hz to 150 Hz while filtering out out-of-band frequencies. The frequency band may vary, and may include, for example, frequencies less than 300 Hz, such as less than 200 Hz, including less than 150 Hz. The passing frequency signals are amplified by an amplifier 2842. Also shown is an accelerometer 2850 electrically coupled to a second multiplexer 2860. The second multiplexer 2860 multiplexes the signals from the accelerometer 2850 with the amplified signals from the amplifier 2842. The multiplexed signals are then converted to digital signals by a converter 2864 which is also electrically coupled to low power processor 2870.

In one aspect, a digital accelerometer (such as one manufactured by Analog Devices), may be implemented in place of the accelerometer 2850. Various advantages may be achieved by using a digital accelerometer. For example, because the signals the digital accelerometer would produce signals already in digital format, the digital accelerometer could bypass the converter 2864 and electrically couple to a low power microcontroller 2870—in which case multiplexer 2860 would no longer be required. Also, the digital signal may be configured to turn itself on when detecting motion, further conserving power. In addition, continuous step counting may be implemented. The digital accelerometer may include a FIFO buffer to help control the flow of data sent to the low power processor 2870. For instance, data may be buffered in the FIFO until full, at which time the processor may be triggered to turn awaken from an idle state and receive the data.

The low power processor 2870 may be, for example, an MSP430 microcontroller from Texas Instruments. The low power processor 2870 of the receiver 2800 maintains the idle state, which as stated earlier, requires minimal current draw—e.g., 10 μA or less, or 1 μA or less.

The high power processor 2880 may be, for example, a VC5509 digital signal process from Texas Instruments. The high power processor 2880 performs the signal processing actions during the active state. These actions, as stated earlier, require larger amounts of current than the idle state—e.g., currents of 30 μA or more, such as 50 μA or more—and may include, for example, actions such as scanning for conductively transmitted signals, processing conductively transmitted signals when received, obtaining and/or processing physiologic data, etc.

The receiver 2800 (e.g., body-associated personal communicator 2104) may include a hardware accelerator module to process data signals. The hardware accelerator module may be implemented instead of, for example, a DSP. Being a more specialized computation unit, it performs aspects of the signal processing algorithm with fewer transistors (less cost and power) compared to the more general purpose DSP. The blocks of hardware may be used to “accelerate” the performance of important specific function(s). Some architectures for hardware accelerators may be “programmable” via microcode or VLIW assembly. In the course of use, their functions may be accessed by calls to function libraries.

The hardware accelerator (HWA) module comprises an HWA input block to receive an input signal that is to be processed and instructions for processing the input signal; and, an HWA processing block to process the input signal according to the received instructions and to generate a resulting output signal. The resulting output signal may be transmitted as needed by an HWA output block.

Also shown in FIG. 25 is a flash memory 2890 electrically coupled to the high power processor 2880. In one aspect, a flash memory 2890 may be electrically coupled to the low power processor 2870, which may provide for better power efficiency.

A wireless communication element 2895 is shown electrically coupled to the high power processor 2880 and may include, for example, a BLUETOOTH (™) wireless communication transceiver. In one aspect, the wireless communication element 2895 is electrically coupled to the high power processor 2880. In another aspect, the wireless communication element 2895 is electrically coupled to the high power processor 2880 and low power processor 2870. Furthermore, wireless communication element 2895 may be implemented to have its own power supply so that it may be turned on and off independently from other components of the receiver—e.g., by a microprocessor.

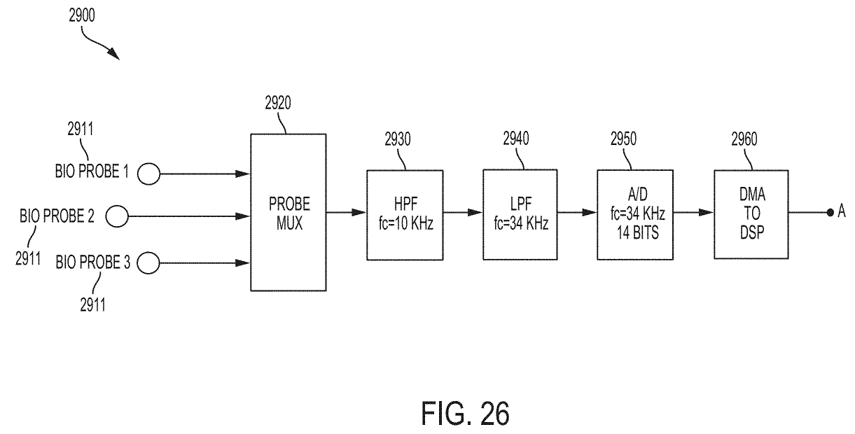

FIG. 26 provides a view of a block diagram of hardware in a receiver 2900 (e.g., body-associated personal communicator 2104) according to one embodiment related to the high frequency signal chain. In FIG. 26, the receiver 2900 includes receiver probes (for example in the form of electrodes 2911, 2912, 2913) electrically coupled to a multiplexer 2920. Also shown are a high pass filter 2930 and a low pass filter 2940 to provide for a band pass filter, which eliminates any out-of-band frequencies. In the aspect shown, a band pass of 10 KHz to 34 KHz is provided to pass carrier signals falling within the frequency band. Example carrier frequencies may include, but are not limited to, 12.5 KHz and 20 KHz. One or more carriers may be present. In addition, the receiver 2900 includes an analog to digital converter 2950, for example, sampling at 500 KHz. The digital signal can thereafter be processed by the DSP. Shown in this aspect is a DMA-to-DSP unit 2960, which sends the digital signal to dedicated memory for the DSP. The direct memory access provides the benefit of allowing the rest of the DSP to remain in a low power mode.

### Example Configurations for Various States

As stated earlier, for each receiver state, the high power functional block may be cycled between active and inactive states accordingly. Also, for each receiver state, various receiver elements (such as circuit blocks, power domains within processor, etc.) of a receiver may be configured to independently cycle from on and off by the power supply module. Therefore, the receiver may have different configurations for each state to achieve power efficiency.

In certain aspects, the receivers are part of a body-associated system or network of devices, such as sensors, signal receivers, and optionally other devices, which may be internal and/or external, which provide a variety of different types of information that is ultimately collected and processed by a processor, such as an external processor, which then can provide contextual data about a living subject, such as a patient, as output. For example, the receiver may be a member of an in-body network of devices which can provide an output that includes data about IEM ingestion, one or more physiologic sensed parameters, implantable device operation, etc., to an external collector of the data. The external collector, e.g., in the form of a health care network server, etc., of the data then combines this receiver provided data with additional relevant data about the patient, e.g., weight, weather, medical record data, etc., and may process this disparate data to provide highly specific and contextual patient specific data.

### Systems

Systems of the disclosure include, in certain aspects, a signal receiver aspect of a receiver and one or more IEMs. IEMs of interest include those described in PCT application serial no. PCT/US2006/016370 published as WO/2006/116718; PCT application serial no. PCT/US2007/082563 published as WO/2008/052136; PCT application serial no. PCT/US2007/024225 published as WO/2008/063626; PCT application serial no. PCT/US2007/022257 published as WO/2008/066617; PCT application serial no. PCT/US2008/052845 published as WO/2008/095183; PCT application serial no. PCT/US2008/053999 published as WO/2008/101107; PCT application serial no. PCT/US2008/056296 published as WO/2008/112577; PCT application serial no. PCT/US2008/056299 published as WO/2008/112578; and PCT application serial no. PCT/US2008/077753 published as WO 2009/042812; the disclosures of which applications are herein incorporated by reference.

In certain aspects the systems include an external device which is distinct from the receiver (which may be implanted or topically applied in certain aspects), where this external device provides a number of functionalities. Such an external device can include the capacity to provide feedback and appropriate clinical regulation to the patient. Such a device can take any of a number of forms. For example, the device can be configured to sit on the bed next to the patient, e.g., a bedside monitor. Other formats include, but are not limited to, PDAs, smart phones, home computers, etc.

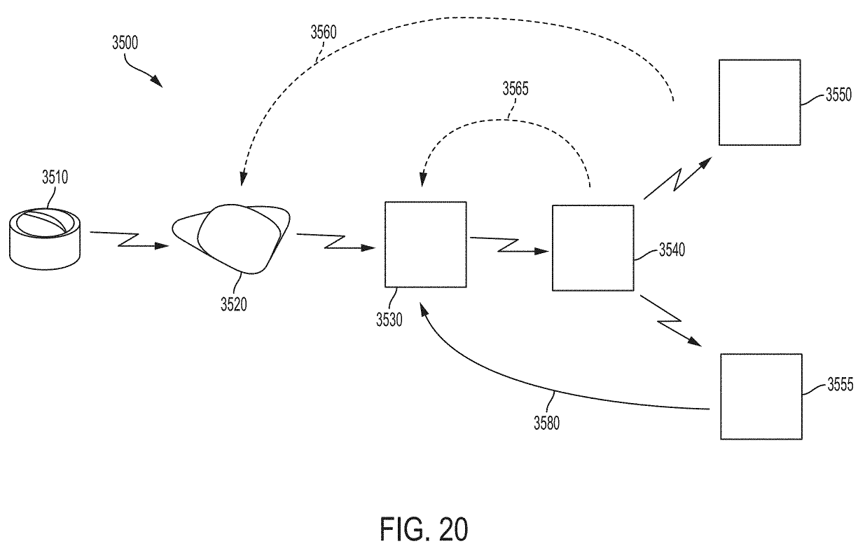

An example of a system is shown in FIG. 20. In FIG. 20, system 3500 includes a pharmaceutical composition 3510 that comprises an IEM. Also present in system 3500 is signal receiver 3520, such as the signal receiver illustrated in FIG. 21. Signal receiver 3520 is configured to detect a signal emitted from the identifier of the IEM 3510. Signal receiver 3520 also includes physiologic sensing capability, such as ECG and movement sensing capability. Signal receiver 3520 is configured to transmit data to a patient's an external device or PDA 3530 (such as a smart phone or other wireless communication enabled device), which in turn transmits the data to a server 3540. Server 3540 may be configured as desired, e.g., to provide for patient directed permissions. For example, server 3540 may be configured to allow a family caregiver 3550 to participate in the patient's therapeutic regimen, e.g., via an interface (such as a web interface) that allows the family caregiver 3550 to monitor alerts and trends generated by the server 3540, and provide support back to the patient, as indicated by arrow 3560. The server 3540 may also be configured to provide responses directly to the patient, e.g., in the form of patient alerts, patient incentives, etc., as indicated by arrow 3565 which are relayed to the patient via PDA 3530. Server 3540 may also interact with a health care professional (e.g., RN, physician) 3555, which can use data processing algorithms to obtain measures of patient health and compliance, e.g., wellness index summaries, alerts, cross-patient benchmarks, etc., and provide informed clinical communication and support back to the patient, as indicated by arrow 1580.

### Re-Wearable Wireless Device

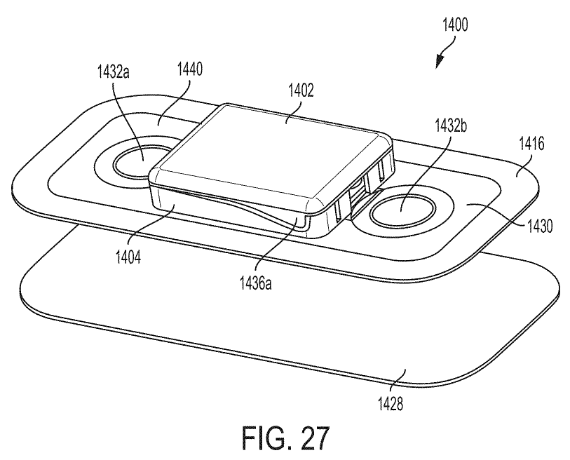

FIGS. 27-34 are illustrations of a re-wearable wireless device 1400 with a switch comprising a metal dome and a compliant actuator, according to one embodiment. The re-wearable wireless device 1400 also includes a pogo pin arrangement to provide electrical contact between a strip and a receiver. The re-wearable wireless devoice also includes a skin adhesive skirt with an oversized release layer on a bottom portion thereof.

FIG. 27 is a perspective view of the re-wearable wireless device 1400 with a removable liner 1428 removed from an adhesive layer 1416, according to one embodiment. The removable liner 1428 is shown as a one piece liner. In other embodiments, the removable liner 1428 may be formed of two or more pieces. The re-wearable wireless device 1400 comprises a reusable component 1402, which may referred to as a pod and a disposable component 1404, which may be referred to as a cradle and is configured to receive the reusable component 1402. The reusable component 1402 also defines a housing to hold various electronic circuits such as receiver and communication circuits described in connection with FIGS. 22-26, for example. The housing of the reusable component 1402 includes a protective guard 1436a on either side to protect electrical interconnection components inside the housing. The housing portions of the reusable component 1402 and disposable component 1404 may be made of plastic. The re-wearable wireless device 1400 also includes a flexible skin adhesive layer 1416. Electrodes 1432a, 1432b and the disposable component 1404 are disposed on a substrate 1440 that is covered with a cover layer 1430. The cover layer 1430 is disposed over the electrodes 1432a, 1432b. The cover layer 1430 may be made of the same material as the skin adhesive.

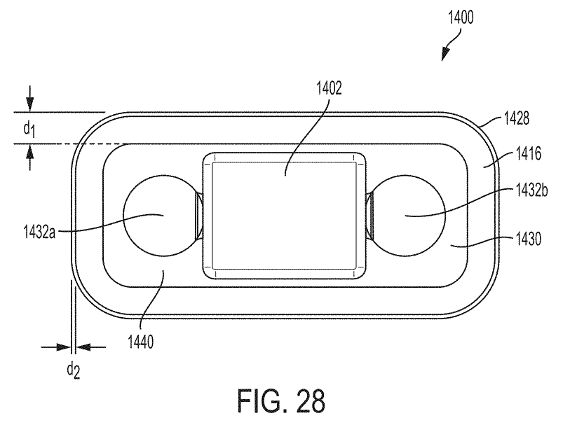

FIG. 28 is a top view of the re-wearable wireless 1400 shown in FIG. 27, according to one embodiment. The re-wearable wireless device 1400 includes a removable liner 1428 that is attached to the adhesive layer underneath the substrate 1440. The flexible skin adhesive layer 1416 is provided at a distance from the disposable component 1404 (FIG. 27) and the reusable component 1402 of the device 1400. The width of the skin adhesive layer 1416 is d₁ and can be anywhere from 4-8 mm or 5-7 mm or preferably about 6 mm. The removable liner 1428 may have a dimension that is slightly larger than and extends outwardly from the footprint of the skin adhesive layer 1416. This dimension is sown as d₂ and may be 1-3 mm or 1.5-2.5 mm or preferably about 1 mm. The extra lateral dimensions of the removable liner 1428 protect the skin adhesive layer 1416 from lifting away from the removable liner 1428.

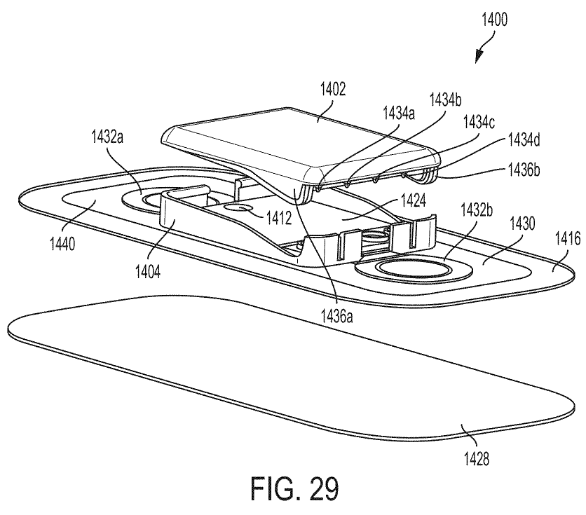

FIG. 29 is an explode view of the reusable component 1402 of the re-wearable wireless device 1400 shown in FIG. 27, according to one embodiment. As shown, the housing of the reusable component 1402 includes electrical contact elements 1434a, 1434b, 1434c, 1434d, for example. The electrical contact elements 1434a, 1434b may be employed to electrically connect the electronic circuitry within the reusable component 1402 to a battery and the electrical contacts 1434c, 1434d may be employed to electrically connect the circuitry to the electrodes 1432a, 1432b. The housing portion of the reusable component 1402 includes protective guards 1436a, 1436 that serve a dual purpose. One purpose is to protect the electrical contact elements 1434a-1434d and due to the asymmetry of the protective guards 1436a, 1436b, they also function as an insertion key such that the reusable component 1402 can only be mated with the disposable component 1404 in one way. Also shown in FIG. 29, is a metal dome 1412 that is actuated by a compliant actuator in the reusable component 1402. The metal dome 1412 when actuated provides electrical contact to connect the battery to the circuits. When the metal dome 142 is not actuated, the battery is disconnected, this saving battery life and minimizing opportunities for unintentionally connecting the battery. The metal dome 1412 and other elements inside the disposable component 1404 are coated with a thin plastic film 1424.

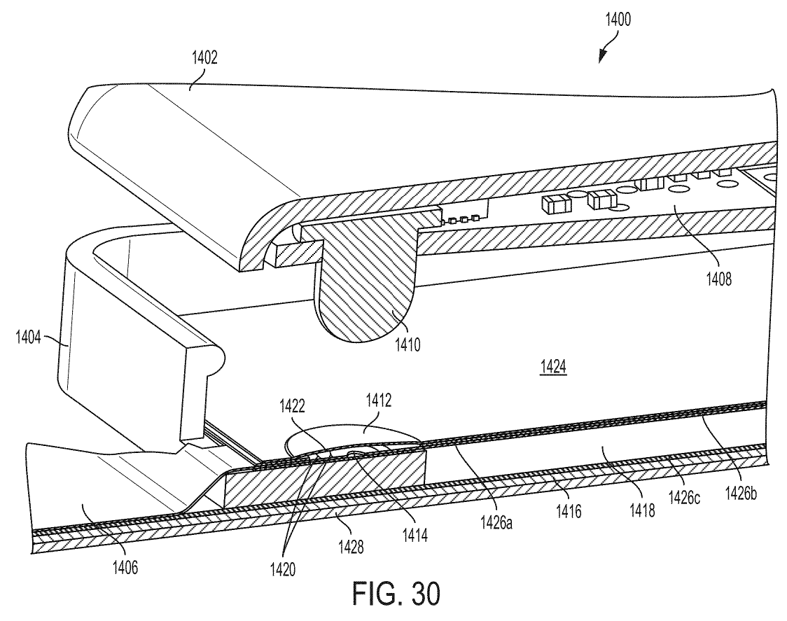

FIG. 30 is an illustration of a perspective view of the reusable component 1402 and disposable component 1404 of the re-wearable wireless device 1400 shown in FIG. 27 prior to mating the two components 1402, 1404, according to one embodiment. As shown in FIG. 30, the reusable component 1402 includes a housing that contains electronic circuits 1408 for a receiver and wireless communication device as described in connection with FIGS. 22-26 hereinabove, which will not be repeated here for conciseness and clarity of disclosure. The reusable component 1402 includes a compliant actuator 1410 which actuates a switch comprising a metal dome 1412 when it lowered onto it. Below the metal dome 1412 is a circuit element 1414 with electrical traces 1420 which are shorted when the metal dome 1412 is flattened or forced into contact with the circuit element 1414. The metal dome 1412 is covered by a thin plastic film 1424. The switch may be mechanical or electrical, and includes any suitable magnetic, electromagnetic, reed, solid state, or other suitable switching element.

The battery is contained in an opening or aperture referred to herein as a battery compartment 1418. The compliant actuator 1410 contacts the metal dome 1412 component of the switch when it is in a mated configuration with the disposable component 1404. Accordingly, when the reusable component 1402 is not inserted or received within the cradle housing of the disposable component 1404, the battery is open circuit for better shelf life.

A flexible circuit 1406 extends to the electrodes 1432a, 1432b (FIGS. 27-29) and is received below the thin plastic film 1424. The thin plastic film 1424 may be formed of any suitable polymeric materials. The compliant actuator 1410 is formed of a compliant deformable material to absorb the mechanical tolerance stack. Thus, the compliant actuator 1410 can be made slightly larger to ensure that adequate force is applied to the metal dome 1412 to make good electrical contact.

Various tie layers 1426a, 1426b, 1426c are used to fasten the various elements of the re-wearable wireless device 1400. The first tie layer 1426a may comprise a double sided sticky tape to fasten or connect the thin plastic film 1424 to the metal dome 142. The flexible circuit 1406 is disposed between the first and second tie layer 1426b and the second tie layer 1426b couples the flexible circuit 1406 to the plastic housing portion of the disposable component 1404. The third tie layer 1426c couples the housing portion of the disposable component 1404 to the skin adhesive layer 1416. The removable liner 1428 is coupled to the skin adhesive layer 1416. The skin adhesive layer 1426 or strip is used to attach the re-usable wireless device 1400 to a user.

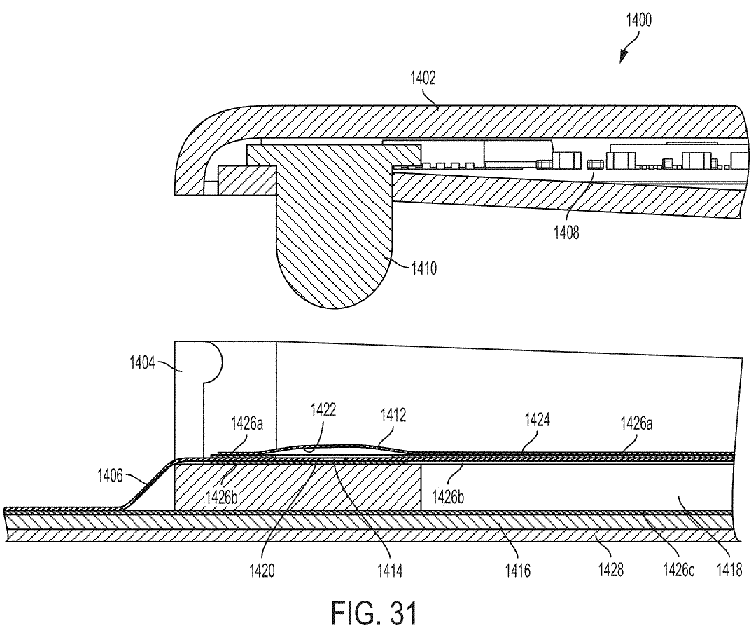

FIG. 31 is a side view of the reusable component 1402 and disposable component 1404 of the re-wearable wireless device 1400 shown in FIG. 27 prior to mating the two components 1402, 1404, according to one embodiment. As shown in FIG. 31, the compliant actuator is not in contacting relationship with the metal dome 1412. Thus, the metal portion 1422 of the metal dome 1410 is in an open position and the battery is open circuit.

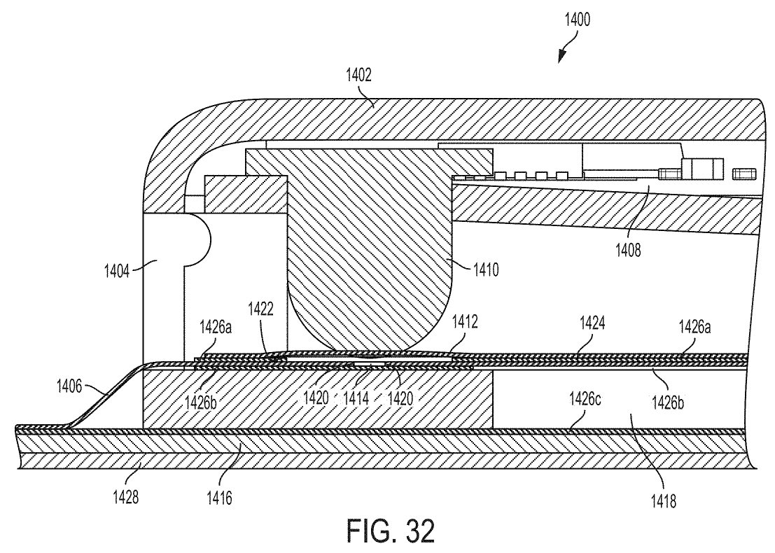

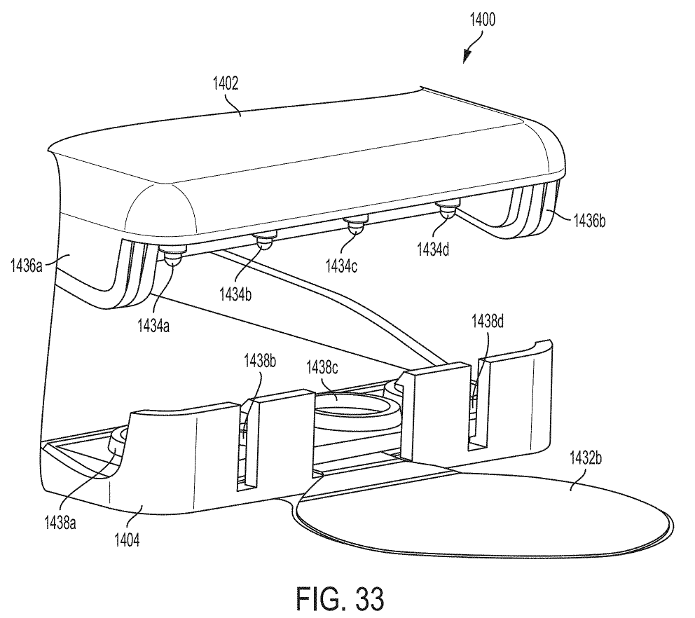

FIG. 32 is a side view of the reusable component 1402 and disposable component 1404 of the re-wearable wireless device 1400 shown in FIG. 27 after mating the two components 1402, 1404, according to one embodiment. As shown in FIG. 32, the compliant actuator is in contacting relationship with the metal dome 1412 and applies a force to flatten or actuate the metal dome 1412 to force the metal portion 1422 of the contact dome 1412 into electrical contact with the circuit element 1414 to short circuit the traces 1420 to create an electrical contact and connect the battery.

FIG. 33 is a detail view of the electrical contact elements 1434a-1434b located within the re-usable component 1402 housing of the re-wearable wireless device 1400 shown in FIG. 27, according to one embodiment. The electrical contact elements 1434a, 1434b, 1434c, 1434c are received within corresponding wells 1438a, 1438b, 1438c, 1438d to make contact with the flexible circuit 1406 (FIGS. 30-32) when the top re-usable component 1402 is received within the disposable component 1404. In one embodiment, the electrical contact elements 1434a, 1434b are employed to electrically connect the battery to the electronic circuits 1408 (FIGS. 30-32) and the contact elements 1434c, 1434d are employed to electrically connect the electronic circuits 1408 to the electrodes 1432a, 1432b. The housing portion of the reusable component 1402 includes protective guards 1436a, 1436 that serve a dual purpose. One purpose is to protect the electrical contact elements 1434a-1434d and due to the asymmetry of the protective guards 1436a, 1436b, they also function as an insertion key such that the reusable component 1402 can only be mated with the disposable component 1404 in one way.

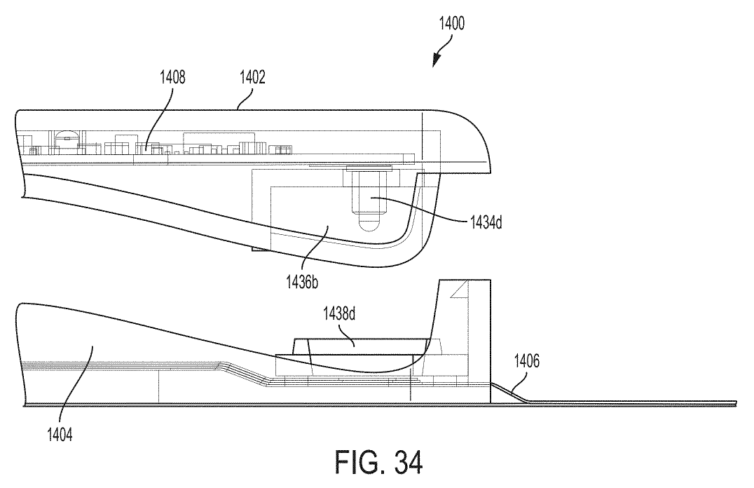

FIG. 34 is side view of the electrical contact elements 1434a-1434b located within the re-usable component 1402 housing of the re-wearable wireless device 1400 shown in FIG. 27, according to one embodiment. As shown, the electrical contact element 1434d is protected by the protective guard 1436b. The electrical contact elements 1434a-1434d connect to the electronic circuits 1408. In one embodiment, the electrical contact elements 1434a-1434d are spring loaded pogo pins. Other suitable contact elements, however, may be employed without limitation.

While various details have been set forth in the foregoing description, it will be appreciated that the various aspects of the loose wearable system may be practiced without these specific details. For example, for conciseness and clarity selected aspects have been shown in block diagram form rather than in detail. Some portions of the detailed descriptions provided herein may be presented in terms of instructions that operate on data that is stored in a computer memory. Such descriptions and representations are used by those skilled in the art to describe and convey the substance of their work to others skilled in the art. In general, an algorithm refers to a self-consistent sequence of steps leading to a desired result, where a “step” refers to a manipulation of physical quantities which may, though need not necessarily, take the form of electrical or magnetic signals capable of being stored, transferred, combined, compared, and otherwise manipulated. It is common usage to refer to these signals as bits, values, elements, symbols, characters, terms, numbers, or the like. These and similar terms may be associated with the appropriate physical quantities and are merely convenient labels applied to these quantities.

Unless specifically stated otherwise as apparent from the foregoing discussion, it is appreciated that, throughout the foregoing description, discussions using terms such as “processing” or “computing” or “calculating” or “determining” or “displaying” or the like, refer to the action and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

It is worthy to note that any reference to “one aspect,” “an aspect,” “one embodiment,” or “an embodiment” means that a particular feature, structure, or characteristic described in connection with the aspect is included in at least one aspect. Thus, appearances of the phrases “in one aspect,” “in an aspect,” “in one embodiment,” or “in an embodiment” in various places throughout the specification are not necessarily all referring to the same aspect. Furthermore, the particular features, structures or characteristics may be combined in any suitable manner in one or more aspects.

Although various embodiments have been described herein, many modifications, variations, substitutions, changes, and equivalents to those embodiments may be implemented and will occur to those skilled in the art. Also, where materials are disclosed for certain components, other materials may be used. It is therefore to be understood that the foregoing description and the appended claims are intended to cover all such modifications and variations as falling within the scope of the disclosed embodiments. The following claims are intended to cover all such modification and variations.

Some or all of the embodiments described herein may generally comprise technologies for various aspects of loose wearable system, or otherwise according to technologies described herein. In a general sense, those skilled in the art will recognize that the various aspects described herein which can be implemented, individually and/or collectively, by a wide range of hardware, software, firmware, or any combination thereof can be viewed as being composed of various types of “electrical circuitry.” Consequently, as used herein “electrical circuitry” includes, but is not limited to, electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of random access memory), and/or electrical circuitry forming a communications device (e.g., a modem, communications switch, or optical-electrical equipment). Those having skill in the art will recognize that the subject matter described herein may be implemented in an analog or digital fashion or some combination thereof.

The foregoing detailed description has set forth various embodiments of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, or examples can be implemented, individually and/or collectively, by a wide range of hardware, software, firmware, or virtually any combination thereof. In one embodiment, several portions of the subject matter described herein may be implemented via Application Specific Integrated Circuits (ASICs), Field Programmable Gate Arrays (FPGAs), digital signal processors (DSPs), or other integrated formats. Those skilled in the art will recognize, however, that some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. In addition, those skilled in the art will appreciate that the mechanisms of the subject matter described herein are capable of being distributed as a program product in a variety of forms, and that an illustrative embodiment of the subject matter described herein applies regardless of the particular type of signal bearing medium used to actually carry out the distribution. Examples of a signal bearing medium include, but are not limited to, the following: a recordable type medium such as a floppy disk, a hard disk drive, a Compact Disc (CD), a Digital Video Disk (DVD), a digital tape, a computer memory, etc.; and a transmission type medium such as a digital and/or an analog communication medium (e.g., a fiber optic cable, a waveguide, a wired communications link, a wireless communication link (e.g., transmitter, receiver, transmission logic, reception logic, etc.), etc.).

All of the above-mentioned U.S. patents, U.S. patent application publications, U.S. patent applications, foreign patents, foreign patent applications, non-patent publications referred to in this specification and/or listed in any Application Data Sheet, or any other disclosure material are incorporated herein by reference, to the extent not inconsistent herewith. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

One skilled in the art will recognize that the herein described components (e.g., operations), devices, objects, and the discussion accompanying them are used as examples for the sake of conceptual clarity and that various configuration modifications are contemplated. Consequently, as used herein, the specific exemplars set forth and the accompanying discussion are intended to be representative of their more general classes. In general, use of any specific exemplar is intended to be representative of its class, and the non-inclusion of specific components (e.g., operations), devices, and objects should not be taken limiting.

With respect to the use of substantially any plural and/or singular terms herein, those having skill in the art can translate from the plural to the singular and/or from the singular to the plural as is appropriate to the context and/or application. The various singular/plural permutations are not expressly set forth herein for sake of clarity.

The herein described subject matter sometimes illustrates different components contained within, or connected with, different other components. It is to be understood that such depicted architectures are merely exemplary, and that in fact many other architectures may be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected,” or “operably coupled,” to each other to achieve the desired functionality, and any two components capable of being so associated can also be viewed as being “operably couplable,” to each other to achieve the desired functionality. Specific examples of operably couplable include but are not limited to physically mateable and/or physically interacting components, and/or wirelessly interactable, and/or wirelessly interacting components, and/or logically interacting, and/or logically interactable components.

Some aspects may be described using the expression “coupled” and “connected” along with their derivatives. It should be understood that these terms are not intended as synonyms for each other. For example, some aspects may be described using the term “connected” to indicate that two or more elements are in direct physical or electrical contact with each other. In another example, some aspects may be described using the term “coupled” to indicate that two or more elements are in direct physical or electrical contact. The term “coupled,” however, also may mean that two or more elements are not in direct contact with each other, but yet still co-operate or interact with each other.

In some instances, one or more components may be referred to herein as “configured to,” “configurable to,” “operable/operative to,” “adapted/adaptable,” “able to,” “conformable/conformed to,” etc. Those skilled in the art will recognize that “configured to” can generally encompass active-state components and/or inactive-state components and/or standby-state components, unless context requires otherwise.

While particular aspects of the present subject matter described herein have been shown and described, it will be apparent to those skilled in the art that, based upon the teachings herein, changes and modifications may be made without departing from the subject matter described herein and its broader aspects and, therefore, the appended claims are to encompass within their scope all such changes and modifications as are within the true spirit and scope of the subject matter described herein. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations.

In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that typically a disjunctive word and/or phrase presenting two or more alternative terms, whether in the description, claims, or drawings, should be understood to contemplate the possibilities of including one of the terms, either of the terms, or both terms unless context dictates otherwise. For example, the phrase “A or B” will be typically understood to include the possibilities of “A” or “B” or “A and B.”

With respect to the appended claims, those skilled in the art will appreciate that recited operations therein may generally be performed in any order. Also, although various operational flows are presented in a sequence(s), it should be understood that the various operations may be performed in other orders than those which are illustrated, or may be performed concurrently. Examples of such alternate orderings may include overlapping, interleaved, interrupted, reordered, incremental, preparatory, supplemental, simultaneous, reverse, or other variant orderings, unless context dictates otherwise. Furthermore, terms like “responsive to,” “related to,” or other past-tense adjectives are generally not intended to exclude such variants, unless context dictates otherwise.

In certain cases, use of a system or method may occur in a territory even if components are located outside the territory. For example, in a distributed computing context, use of a distributed computing system may occur in a territory even though parts of the system may be located outside of the territory (e.g., relay, server, processor, signal-bearing medium, transmitting computer, receiving computer, etc. located outside the territory).

A sale of a system or method may likewise occur in a territory even if components of the system or method are located and/or used outside the territory. Further, implementation of at least part of a system for performing a method in one territory does not preclude use of the system in another territory.

Although various embodiments have been described herein, many modifications, variations, substitutions, changes, and equivalents to those embodiments may be implemented and will occur to those skilled in the art. Also, where materials are disclosed for certain components, other materials may be used. It is therefore to be understood that the foregoing description and the appended claims are intended to cover all such modifications and variations as falling within the scope of the disclosed embodiments. The following claims are intended to cover all such modification and variations.

In summary, numerous benefits have been described which result from employing the concepts described herein. The foregoing description of the one or more embodiments has been presented for purposes of illustration and description. It is not intended to be exhaustive or limiting to the precise form disclosed. Modifications or variations are possible in light of the above teachings. The one or more embodiments were chosen and described in order to illustrate principles and practical application to thereby enable one of ordinary skill in the art to utilize the various embodiments and with various modifications as are suited to the particular use contemplated. It is intended that the claims submitted herewith define the overall scope.

What is claimed is:

1. A re-wearable wireless device, configured to be attached to a user, the re-wearable device comprising:
a disposable component, wherein the disposable component comprises:
a first electrode;
a second electrode;
a cradle;
a battery;
wherein the cradle comprises a first electrical connector, wherein the first electrical connector is electrically coupled to the first electrode, the second electrode, and the battery; and
a reusable component, wherein the reusable component comprises:
an electronics module;
a housing configured to latch into the cradle;
a second electrical connector, wherein the second electrical connector electrically couples the reusable component to the first electrode, the second electrode, and the battery;
wherein the re-wearable wireless device is configured to detect a conductive electrical signal through the first electrode and the second electrode.

2. The re-wearable wireless device of claim 1, wherein the disposable component is configured to be removably attached to a user, and
wherein the first electrode and the second electrode are configured to couple to an external body portion of the user.

3. The re-wearable wireless device of claim 1, wherein the disposable component further comprises:
an adhesive patch, wherein the first electrode and the second electrode are attached to the adhesive patch in a way that enables the first and second electrodes to couple to an external body portion of the user.

4. The re-wearable wireless device of claim 3, wherein the adhesive patch comprises a skirt of free adhesive material around the cradle of the disposable component.

5. The re-wearable wireless device of claim 4, wherein the skirt comprises a width between 4 mm and 8 mm.

6. The re-wearable wireless device of claim 1, wherein:
the disposable component further comprises a switch connected between the battery and at least one electrical connector of the cradle,
the reusable component further comprise an actuator, wherein the actuator activates the switch
when the reusable component is connected to the disposable component, and
the battery is disconnected from the at least one electrical connector of the cradle when the switch is not activated.

7. The re-wearable wireless device of claim 6, wherein the switch is mechanical.

8. The re-wearable wireless device of claim 6, wherein the switch is electrical.

9. The re-wearable wireless device of claim 6, wherein the switch comprises a magnetic element.

10. The re-wearable wireless device of claim 6, wherein the switch comprises an electromagnetic element.

11. The re-wearable wireless device of claim 6, wherein the switch comprises a reed switching element.

12. The re-wearable wireless device of claim 6, wherein the switch comprises a solid state switching element.

13. The re-wearable wireless device of claim 6, wherein:
the switch comprises a dome switch, and
the actuator is configured to press on the dome switch when the reusable component is connected to the disposable component.

14. The re-wearable wireless device of claim 13, wherein the dome switch is made of metal and further comprises a circuit element with electrical traces located below the dome, and wherein the electrical traces are shorted when the actuator presses on the dome switch.

15. The re-wearable wireless device of claim 13, wherein the actuator is formed of a compliant material.

16. The re-wearable wireless device of claim 1, wherein:
the reusable component comprises a housing,
the housing comprises at least one protective guard located along at least one edge of the reusable component,
the cradle comprises at least one mating feature configured to mate with the at least one protective guard, and
the at least one protective guard is configured in such a way that the reusable component can only be latched on to the disposable component in one orientation of the reusable component with respect to the disposable component.

17. The re-wearable wireless device of claim 16, wherein:
the at least one protective guard comprises a protruding wall extending along an edge of the reusable component,
the protruding wall has a free edge,
the free edge comprises a first end and a second end, and
a distance between the free edge and the base of the protruding wall is lower at the first end than it is at the second end.

18. The re-wearable wireless device of claim 1, wherein the at least one connector in the reusable component comprises at least one spring-loaded pogo pin.

19. The re-wearable wireless device of claim 1, wherein:
the cradle of the disposable component comprises at least one well disposed around each of the at least one electrical connector, and
each of the at least one well forms an enclosure around the at least one electrical connector in the reusable component and its corresponding electrical connector in the disposable component when the reusable component is latched onto the disposable component.

20. The re-wearable wireless device of claim 1, wherein the conductive electrical signal is produced by an ingestible device upon ingestion by the user.

21. The re-wearable wireless device of claim 1, wherein the conductive electrical signal is naturally generated by the user's body.

22. The re-wearable wireless device of claim 1, wherein the reusable component comprises at least one wearer interface.

23. The re-wearable wireless device of claim 22, wherein the at least one wearer interface comprises at least one of the following: a light source, a vibration motor, a touch sensor, a tap sensor, and a speaker.

24. The re-wearable wireless device of claim 1, wherein the reusable component further comprises at least one sensor.

25. The re-wearable wireless device of claim 24, wherein the at least one sensor comprises at least one of the following: a thermistor, an ambient light sensor, a pressure sensor, an accelerometer, a blood pressure sensor, a passive infrared sensor, a hydration sensor, a breathing sensor, a microphone, and a capacitive proximity sensor.

26. The re-wearable wireless device of claim 1, wherein the reusable component comprises a communication module.

27. The re-wearable wireless device of claim 1, wherein the communication module comprises one or more of the following: a Bluetooth module, a cellular modem, a wireless antenna module, a GPS module, and a GNSS module.
